SEQUENCE LISTING (6 sequences)

SEQ ID NO: 1 - protein, 34 aa, Homo sapiens
  PEPTIDE 1..34
  PEPTIDE 1..34
SVSEIQLMHNLGKHLNSMERVEWLRKKLQDVHNF

SEQ ID NO: 2 - protein, 34 aa, Rattus rattus
AVSEIQLMHNLGKHLASVERMQWLRKKLQDVHNF

SEQ ID NO: 3 - protein, 12 aa, Homo sapiens
NTQTLAKAPEHT

SEQ ID NO: 4 - protein, 12 aa, Homo sapiens
TYTDWLNFWAWP

SEQ ID NO: 5 - protein, 12 aa, Homo sapiens
KSLSRHDHIHHH

SEQ ID NO: 6 - protein, 11 aa, Homo sapiens
DFTKTSPLGIH

(19) United States
(12) Patent Application Publication
(10) Pub. No.: US 2025/0082204 A1
(43) Pub. Date: Mar. 13, 2025

(54) IMAGING SYSTEMS AND METHODS FOR TISSUE DIFFERENTIATION, E.G., FOR INTRAOPERATIVE VISUALIZATION

(71) Applicants: Memorial Sloan Kettering Cancer Center, New York, NY (US); Cornell University, Ithaca, NY (US)

(72) Inventors: Michelle S. Bradbury, Ithaca, NY (US); Barney Yoo, New York, NY (US); UIrich Wiesner, Ithaca, NY (US); Peiming Chen, Brooklyn, NY (US); Kai Ma, Ithaca, NY (US); Snehal G. Patel, New York, NY (US); Daniella Karassawa Zanoni, New York, NY (US); Joseph H. Dayan, New York, NY (US); Nadeem R. Abu-Rustum, Tenafly, NJ (US)

(21) Appl. No.: 18/630,488

(22) Filed: Apr. 9, 2024

Related U.S. Application Data
(63) Continuation of application No. 16/060,463, filed on Jun. 8, 2018, now abandoned.
(60) Provisional application No. 62/349,538, filed on Jun. 13, 2016.
(60) Provisional application No. 62/267,676, filed on Dec. 15, 2015.

(51) Int. Cl. A61B 5/00; A61K 49/00; A61K 51/08; A61K 51/12; A61M 5/00

(52) U.S. Cl. CPC A61B 5/0071; A61B 5/413; A61B 5/418; A61B 5/743; A61K 49/0002; A61K 49/0032; A61K 49/0056; A61K 49/0058; A61K 49/0067; A61K 49/0093; A61K 51/082; A61K 51/1244; A61M 5/007; A61M 2005/006

(57) ABSTRACT

Described herein is a multiplex platform that uses ultrasmall nanoparticles (e.g., C dots and C′ dots) to graphically differentiate specific nerves (e.g., sensory nerves vs. motor nerves) for nerve transplants and other surgeries. Also described herein is a multiplex platform that uses ultrasmall nanoparticles (e.g., C dots and C′ dots) to graphically differentiate between different types of lymph nodes and/or lymphatic pathways, e.g., to safely and effectively perform vascularized lymph node transplantation in the treatment of lymphedema. Also described herein is a multiplex platform that uses ultrasmall nanoparticles (e.g., C dots and C′ dots) to graphically differentiate parathyroid tissue.

### CROSS-REFERENCE TO RELATED APPLICATIONS

This application claims the benefit of U.S. Provisional Application No. 62/267,676, filed on Dec. 15, 2015 and U.S. Provisional Application No. 62/349,538, filed on Jun. 13, 2016, the contents of which are hereby incorporated by reference herein in their entireties.

### GOVERNMENT SUPPORT

This invention was made with government support under grant number CA199081 awarded by the National Institutes of Health. The government has certain rights in this invention.

### SEQUENCE LISTING

The present specification makes reference to a Sequence Listing (submitted electronically as a .txt file named “SEQUENCE LISTING 2003080-1277.txt” on Dec. 15, 2016). The .txt file was generated on Dec. 13, 2016 and is 1.75 kilobytes in size. The entire contents of the Sequence Listing are hereby incorporated by reference.

### TECHNICAL FIELD

This invention relates generally to methods for graphically differentiating between different lymphatic drainage pathways, and for graphically differentiating between different tissues (e.g., nerves, e.g., parathyroid), e.g., during surgery. More particularly, in certain embodiments, the invention relates to reverse lymphatic multiplex mapping, a multiplexed real-time method for differentiation of lymph nodes during a surgical procedure, e.g., to avoid occurrence of lymphedema, or to identify nodes for transplantation in the treatment of lymphedema. Furthermore, in certain embodiments, the invention relates to visual differentiation between nerves (e.g., sensory vs. motor) for nerve reconstruction and other surgeries.

### BACKGROUND

Nerve degeneration decreases the ability of an operating surgeon to identify nerve structures within the operative field, which may complicate and/or limit surgical repair efforts. Chronic denervation injury from, for instance, cancer resection, leads to unilateral muscle paralysis, which restricts movement and results in functional impediments (i.e., loss of blink reflex). Surgical reconstruction of the nerve can re-establish function. Selection of the appropriate reconstructive approach depends on localization of the defect and timing interval since injury.

For instance, iatrogenic nerve injury following surgery is a highly morbid complication often leading to permanent disability. Iatrogenic nerve injury can lead to paralysis if a motor nerve is involved, or loss of sensation or severe chronic pain if a sensory nerve is involved. The risk of these complications can significantly be reduced if the surgeon can better visualize the nerves in the operative field. As an example, temporary or permanent facial palsy following parotidectomy has been reported to have an incidence of up to 45% and 17%, respectively (J Craniomaxillofac Surg. 2015 Jan. 15. pii: S1010-5182 (15) 00012-8. [Epub ahead of print] Comparison of the effect of total conservative parotidectomy versus superficial parotidectomy in management of benign parotid gland tumor: A systematic review. El Fol HA, Beheiri MJ, Zaqri WA).

The facial nerve branches that power muscles of the face are small and run through the parotid gland, making the nerves vulnerable to injury. Because the facial nerve branches are similar in color to the surrounding tissue, these nerves can be difficult to identify, especially in a bloody field. By using a topical agent with a dye conjugated to antibody fragments specific to motor nerves (e.g., ChAT) and a different colored dye conjugated to antibody fragments specific to all nerves (e.g., NBP), the surgeon can not only clearly identify nerves but can also discriminate between critical motor nerves that must be preserved and sensory nerves which can be sacrificed. However, dyes conjugated to an antibody fragment specific to motor nerves or all nerves are limited by the visibility these compositions provide to the surgeon, and selectivity of these compositions to be taken up by the type of nerve tissue.

Hand surgery is another application where identification of motor versus sensory nerves is important, particularly when performing nerve transfers. For example, the median nerve has distinct motor and sensory units. When attempting to repair a damaged nerve with nerve graft or use a portion of the nerve to improve power to a weak muscle group, it is critical to select the appropriate motor or sensory bundle. This is currently performed by speculating the likely location of these bundles based on topography, but ultimately the surgeon has no certainty.

Additionally, facial reanimation procedures are routinely performed to treat facial paralysis and involve transplantation of both muscle and nerve. These highly technical cases require clear visualization and differentiation between sensory and motor nerves to be successful.

Moreover, vascularized lymph node transplantation involves transferring lymph nodes from one part of the body to the affected limb with lymphedema or in a patient at overwhelming risk for developing lymphedema. One significant challenge of this procedure is that one can cause lymphedema when harvesting lymph nodes from the neck, axilla, or groin. Techniques of reverse lymphatic mapping for lymph node transfer to treat lymphedema have been attempted. However, these techniques rely on radioisotopes (e.g., technetium sulfur colloid) to identify lymph nodes draining the extremities using a gamma probe (e.g., Geiger counter-like device which produces an audio signal). The target lymph nodes using these technologies are mapped using indocyanine green dye, which is not specific and leaks freely into the operative field, thereby obscuring the image required for treatment.

Reverse mapping using technetium and a blue dye has been described for removing axillary lymph nodes for breast cancer treatment. In this scenario, the breast surgeon must solely rely on technetium to identify the sentinel lymph nodes of the breast which has a lower sensitivity than combined dye and technetium and could have the serious consequence of a false negative which would result in leaving a metastatic lymph node in the patient.

Similarly, nerve degeneration decreases the ability of an operating surgeon to identify nerve structures within the operative field, which complicates and/or limits surgical repair efforts. Chronic denervation injury from, for instance, cancer resection, leads to unilateral muscle paralysis, which restricts movement and results in functional impediments (i.e., loss of blink reflex). Surgical reconstruction of the nerve can re-establish function. Selection of the appropriate reconstructive approach depends on localization of the defect and timing interval since injury. However, no technologies exist that easily provide visual differentiation between different nerves. Nerve tissue is difficult for a surgeon to see during surgery, and improperly cutting or damaging nerves during surgery can have a lifelong adverse impact for the patient.

Therefore, there is a need for tissue-binding agents (e.g., nerve-binding agents) with enhanced selectivity to differentiate between different types of tissues (e.g., different types of nerves) during such procedures (e.g., motor versus sensory nerves). Further, there is a need to distinguish critical vs. sensory nerve motor branches during surgeries to facilitate in determining which nerve or portion thereof can be sacrificed during surgical procedures.

Moreover, there remains a need for a sensitive, multiplexed real-time method for lymphatic mapping, e.g., to facilitate lymph node transfer in the surgical treatment of lymphedema. In addition, the need to differentiate between different types of nerves during surgical procedures (e.g., motor versus sensory nerves) is critically important.

### SUMMARY

As described herein, different dyes can be attached to tissue binding peptides (e.g., nerve binding peptides, e.g., parathyroid binding peptides) and/or incorporated within peptide-functionalized nanoparticles (e.g., ultrasmall nanoparticles having a diameter less than 30 nm, less than 20 nm, less than 10 nm; e.g., C or C′ dots) to permit fluorescence-based multiplexing for “tagging” various tissue (e.g., neural) structures. The sequence and/or conformation of the cyclic (or linear) peptide used, either in its native form or attached to the particle may be adjusted for different tissue and/or nerve types, for example, to enable visual differentiation of the nerve types during surgery (e.g., the different nerve types have a different color). This is important during various nerve repair surgeries (e.g., surgery for facial droop), where the surgeon tries to find a normal nerve segment (“good side”) to graft to an affected area (“bad side”). Few surgeons can perform these types of surgeries, as it is difficult to differentiate particular types of nerve tissue needed for grafts. The nerve binding peptide (and/or fluorescent particle) compositions would facilitate/simplify such surgeries by allowing visual differentiation of specific nerve tissue types.

Moreover, described herein is a multiplex platform that uses ultrasmall nanoparticles (e.g., C dots and C′ dots) to graphically differentiate specific nerves (e.g., sensory nerves vs. motor nerves) for nerve transplants and other surgeries. Also described herein is a multiplex platform that uses ultrasmall nanoparticles (e.g., C dots and C′ dots) to graphically differentiate between different types of lymph nodes and/or lymphatic pathways, e.g., to safely and effectively perform vascularized lymph node transplantation in the treatment of lymphedema.

For example, a technique referenced herein as “Reverse Lymphatic Multiplex Mapping (RLMM)” uses ultrasmall nanoparticles (e.g., C dots and/or C′ dots) that fluoresce at two different wavelengths. In certain embodiments, RLMM allows the surgeon to map the lymph nodes which drain the extremities in a manner that graphically differentiates them from lymph nodes which drain the tumor site. This enhanced visualization allows the surgeon to avoid damaging critical lymph nodes that may lead to lymphedema. Furthermore, RLMM using these ultrasmall nanoparticles can be used to safely perform vascularized lymph node transplantation in the treatment of lymphedema (e.g., to identify nodes suitable for transplantation). For example, targeted lymph nodes for lymph node harvest draining the trunk can be identified with a nanoparticle using a different colored dye, allowing the surgeon to cherry pick lymph nodes that will not affect drainage of the adjacent limb. This technique allows for the safe harvest of lymph nodes in lymph node transplantation for lymphedema.

The surgical technique for RLMM is the quite similar for both tumor resection and lymphadenectomy as well as lymph node transplantation, a difference being the location of injection. For tumor removal and lymphadenectomy, nanoparticles of one color are injected into the tumor site which would illuminate the lymph nodes targeted for removal. Nanoparticles of a different color are then injected into the adjacent limb at risk for developing lymphedema. The critical lymphatic vessels and lymph nodes are intensely illuminated in a contrasting color allowing the surgeon to clearly visualize and avoid these lymph nodes, minimizing the risk of iatrogenic lymphedema. For lymph node transplantation, the only difference is the first injection is in the trunk draining the lymph nodes targeted for harvest. (Dayan et al., “Reverse lymphatic mapping: a new technique for maximizing safety in vascularized lymph node transfer.” Plast Reconstr Surg. 2015 January; 135 (1): 277-85) Without this technique, it is challenging for a surgeon to determine which lymph nodes are safe to remove and which can cause permanent disability.

As an example, a patient with a particular cancer who needs axillary lymph nodes removed receives a first injection of a first type of C dot that fluoresces at a first spectrally distinct wavelength, where the first injection is injected into or near a tumor site. The patient also receives a second injection of a second type of C dot that fluoresces at a second wavelength spectrally distinct from the first wavelength, where the second injection is injected into an extremity (e.g., an upper or lower extremity near the tumor site) that would be potentially affected by lymphedema if a lymphatic drainage pathway affecting that extremity is disturbed by removal of a lymph node for that pathway. For example, in the case of melanoma, a first injection site can be at the site of melanoma (e.g., on the trunk, abdomen, pelvis) and the second site would be at the potentially affected extremity. For example, in the case of breast cancer, a first injection site can be the thoracic cavity; and in the case of gynecological cancers, a first injection site can be the pelvic area. The second injection would then be in the extremity that would be potentially affected by lymphedema. Being able to differentiate between the first type and second types of C dots reduces risk of lymphedema to the extremity by avoiding removing the drainage lymph node.

For instance, a patient with breast cancer who needs axillary lymph nodes removed has one type of C dot that fluoresces green which is injected into the breast (e.g., wherein the fluorophore is part of the particle itself or is attached to or otherwise associated with the particle). Another C dot that fluoresces blue (or is otherwise spectrally differentiated from the first C dot) is injected into a potentially affected extremity (e.g., the lower or the upper limb), e.g., an extremity near the tumor site. For example, when removing the axillary nodes, the surgeon can specifically remove only green lymph nodes draining the breast and avoid blue lymph nodes draining the upper limb. The imaging technique can be performed as part of a surgical procedure, or it may be performed for pre-surgical imaging. This technique can be performed with any cancer where a node is removed or transplanted.

As another example, RLMM allows the surgeon to reduce the risk in operations involving nerves and consequences of nerve damage, particularly facial nerve damage. For example, a first type of nanoparticle with ligands attached that facilitate (at least temporary) binding of the nanoparticle to motor nerves are administered to a patient, and a second type of nanoparticle with ligands attached that facilitate binding of the nanoparticle to sensory nerves are administered to the patient, wherein the first and second type of nanoparticles are spectrally distinguishable from each other. Examples of ligands for binding of nanoparticles to specific nerve types are described in U.S. Provisional Application No. 62/267,676 “Compositions comprising cyclic peptides, and use of same for visual differentiation of nerve tissue during surgical procedures.”, attached hereto and incorporated herein by reference in its entirety. During surgery, motor nerves fluoresce one color (e.g., green) while sensory nerves fluoresce another color (e.g., blue), providing the surgeon with enhanced ability to identify different nerves and/or avoid cutting certain nerves. The technique may be useful in both surgical settings and non-surgical (e.g., pre-surgical imaging) settings.

The RLMM technology described in this application maintains a high sensitivity as well as reducing the risk of causing lymphedema or additional nerve during these procedures.

In one aspect, the invention is directed to a method comprising: administering two or more different probe species each comprising a spectrally differentiable fluorescent reporter to a lymphatic system; and directing excitation light into the lymph nodes, thereby exciting the fluorescent reporters having spectrally distinguishable emission wavelengths.

In certain embodiments, the administering comprises intravenously administering two or more different probe species. In certain embodiments, the two or more different probe species comprise nanoparticles. In certain embodiments, at least a first probe is administered to a tumor site and at least a second probe is administered to an extremity that would be potentially affected by lymphedema. In certain embodiments, the tumor site comprises a member selected from the group consisting of a breast, a trunk, an abdomen, a pelvis, and a thoracic cavity. In certain embodiments, the extremity comprises a member selected from the group consisting of an upper limb and a lower limb.

In certain embodiments, the excitation light comprises two or more wavelengths, thereby exciting the different fluorescent reporters.

In certain embodiments, the method comprises identifying an appropriate lymph node for transplantation in the treatment of lymphedema.

In certain embodiments, the method comprises simultaneously detecting fluorescent light of spectrally different emission wavelengths, the detected fluorescent light having been emitted by the fluorescent reporters of the respective probe species in the lymph nodes and/or drainage pathways as a result of illumination by excitation light so as to discriminate between signals received from each probe species.

In certain embodiments, the fluorescent reporter of a first probe species having received the excitation light fluoresces at a spectrally distinguishable wavelength compared to a second fluorescent reporter of another probe species having received the excitation light.

In certain embodiments, a signal comprising the spectrally distinguishable emission wavelengths is represented on a display to graphically distinguish between two kinds of lymph nodes and/or drainage pathways.

In certain embodiments, the method comprises identifying an appropriate lymph node for excision.

In certain embodiments, an upper portion of the display shows a first probe species and the bottom portion of the display shows a second probe species. In certain embodiments, the display shows a superimposed image of the first and second probe species.

In certain embodiments, the method comprises displaying a map of lymph nodes and/or lymphatic pathways of the lymphatic system, wherein the map graphically differentiates between specific lymph nodes and/or between specific lymph node types.

In certain embodiments, at least one lymph node drains the extremities and at least one lymph node drains a tumor site. In certain embodiments, the tumor site comprises a member selected from the group consisting of abreast, a trunk, an abdomen, a pelvis, and a thoracic cavity. In certain embodiments, fluorescent reporter of one probe species indicates drainage to the extremities. In certain embodiments, fluorescent reporter of one probe species indicates drainage to the tumor site, thereby avoiding critical lymph nodes that may lead to lymphedema.

In another aspect, the invention is directed to a method comprising: administering two or more different probe species each comprising a spectrally differentiable fluorescent reporter to nerves; and directing excitation light into the nerves, thereby exciting the fluorescent reporters having spectrally distinguishable emission wavelengths.

In certain embodiments, the administering comprises intravenously administering two or more different probe species.

In certain embodiments, the two or more different probe species comprise nanoparticles.

In certain embodiments, the nerves comprise a member selected from the group consisting of, motor nerves and sensory nerves.

In certain embodiments, at least a first probe is administered to a motor nerve and at least a second probe is administered to a sensory nerve.

In certain embodiments, the excitation light comprises two or more wavelengths, thereby exciting the different fluorescent reporters.

In certain embodiments, the method comprises identifying an appropriate nerve for nerve transplantation or other surgeries.

In certain embodiments, the method comprises simultaneously detecting fluorescent light of spectrally different emission wavelengths, the detected fluorescent light having been emitted by the fluorescent reporters of the respective probe species in the nerves as a result of illumination by excitation light so as to discriminate between signals received from each probe species.

In certain embodiments, the fluorescent reporter of a first probe species having received the excitation light fluoresces at a spectrally distinguishable wavelength compared to a second fluorescent reporter of another probe species having received the excitation light.

In certain embodiments, a signal comprising the spectrally distinguishable emission wavelengths is represented on a display to graphically distinguish between two or more kinds of nerves. In certain embodiments, the method comprises identifying an appropriate nerve for excision. In certain embodiments, an upper portion of the display shows a first probe species and the bottom portion of the display shows a second probe species. In certain embodiments, the display shows a superimposed image of the first and second probe species.

In certain embodiments, the method comprises displaying a map of the nerves, wherein the map visually differentiates between specific nerve types. In certain embodiments, one nerve is a sensory nerve and one nerve is a motor nerve. In certain embodiments, the fluorescent reporter of one probe species indicates a motor nerve. In certain embodiments, the fluorescent reporter of one probe species indicates a sensory nerve, thereby differentiating between types of nerves.

In certain embodiments, the two or more probes species comprise silica. In certain embodiments, the two or more probe species comprise nanoparticles that have a silica architecture and dye-rich core. In certain embodiments, nanoparticles comprise C or C′ dots. In certain embodiments, the dye rich core comprises the fluorescent reporter. In certain embodiments, the fluorescent reporter is a near infrared or far red dye. In certain embodiments, the fluorescent reporter is selected from the group consisting of a fluorophore, fluorochrome, dye, pigment, fluorescent transition metal, and fluorescent protein. In certain embodiments, the fluorescent reporter is selected from the group consisting of Cy5, Cy5.5, Cy2, FITC, TRITC, Cy7, FAM, Cy3, Cy3.5, Texas Red, ROX, HEX, JA133, AlexaFluor 488, AlexaFluor 546, AlexaFluor 633, AlexaFluor 555, AlexaFluor 647, DAPI, TMR, R6G, GFP, enhanced GFP, CFP, ECFP, YFP, Citrine, Venus, YPet, CyPet, AMCA, Spectrum Green, Spectrum Orange, Spectrum Aqua, Lissamine, Europium, Dy800 dye, and LiCor 800 dye.

In certain embodiments, the fluorescent light from the fluorescent reporters are detected and mapped in real-time using a handheld fluorescence camera system.

In another aspect, the invention is directed to a kit comprising: a plurality of containers, wherein each container has a type selected from the group consisting of an ampule, a vial, a cartridge, a reservoir, a lyo-ject, and a pre-filled syringe; a first probe species each comprising a first fluorescent reporter; a second probe species each comprising a second fluorescent reporter, wherein a first container of the plurality of containers holds the first probe species and the second container of the plurality of containers holds the second probe species.

In certain embodiments, the kit is for identification of an appropriate lymph node for excision. In certain embodiments, the kit is for use in treating lymphedema. In certain embodiments, the kit is for identification of an appropriate nerve for transplantation.

In certain embodiments, the nerve comprises a member selected from the group consisting of a motor nerve and sensory nerve.

In certain embodiments, the first and second probe species comprise a member selected from the group consisting of nanoparticles, C dots, and C′ dots. In certain embodiments, the first and second probe species further comprise a first nerve binding peptide and a second nerve binding peptide, respectively.

In certain embodiments, the first and second nerve binding peptides comprise a peptide sequence selected from the group consisting of comprises the peptide sequence NTQTLAKAPEHT (SEQ ID NO: 3), TYTDWLNFWAWP (SEQ ID NO: 4), KSLSRHDHIHHH (SEQ ID NO: 5), and DFTKTSPLGIH (SEQ ID NO: 6).

In another aspect, the invention is directed to an imaging method comprising: administering to a subject a plurality of compositions, each composition comprising at least one peptide, and allowing the compositions to selectively bind to tissues of the subject, wherein a first composition of the plurality comprises a first peptide that selectively binds to a first tissue type and wherein a second composition of the plurality comprises a second peptide that selectively binds to a second tissue type; exposing tissue of the subject to excitation light; and detecting light emitted by a first fluorescent agent of the first composition and a second fluorescent agent of the second composition to create an image and displaying the image.

In certain embodiments, the first tissue type comprises sensory nerve tissue. In certain embodiments, the second nerve tissue type comprises motor nerve tissue.

In certain embodiments, the first tissue type comprises parathyroid tissue.

In certain embodiments, the first tissue type comprises a lymph node.

In certain embodiments, the exposing is performed intraoperatively.

In certain embodiments, light emitted by the first fluorescent agent is distinguishable from light emitted by the second fluorescent agent. In certain embodiments, light emitted by the first fluorescent agent is visually distinguishable from the light emitted by the second fluorescent agent. In certain embodiments, light emitted by the first fluorescent agent has a different color that the light emitted by the second fluorescent agent.

In another aspect, the invention is directed to an imaging method comprising: exposing tissue of a subject to excitation light, wherein the tissue comprises a formulation comprising a tissue-binding composition having been administered to the subject, said tissue-binding composition preferentially binding to a particular tissue type; and detecting light emitted by the fluorescent agent of the composition, thereby visually distinguishing the particular tissue type comprising the tissue-binding composition from surrounding tissue.

In certain embodiments, the particular tissue type is nerve tissue. In certain embodiments, the particular tissue type is lymph node tissue. In certain embodiments, the particular tissue type is parathyroid tissue.

In certain embodiments, the tissue-binding composition comprises: a tissue-binding peptide conjugate comprising a peptide; a nanoparticle; a fluorescent agent; and a linker moiety.

In certain embodiments, the peptide comprises an alpha-helix structure. In certain embodiments, the peptide comprises a member selected from the group consisting of a cyclic peptide and a linear peptide. In certain embodiments, peptide comprises an N-methylated amino acid.

In certain embodiments, the tissue-binding peptide conjugate comprises a member selected from the group consisting of a nerve-binding peptide conjugate, lymph-node binding conjugate, and a parathyroid-binding conjugate.

In certain embodiments, the imaging method differentiates nerve tissue from other tissue.

In certain embodiments, the tissue-binding composition comprises: a linear or cyclic peptide comprising a peptide sequence selected from the group consisting of TQTLAKAPEHT (SEQ ID NO: 3), TYTDWLNFWAWP (SEQ ID NO: 4), SLSRHDHIHHH (SEQ ID NO: 5), and DFTKTSPLGIH (SEQ ID NO: 6).

In certain embodiments, the tissue-binding composition comprises: a nerve-binding peptide conjugate, comprising: a linear or cyclic peptide composition comprising: a fluorescent agent; and a linear or cyclic peptide comprising a peptide sequence selected from the group consisting of NTQTLAKAPEHT (SEQ ID NO: 3), TYTDWLNFWAWP (SEQ ID NO: 4), KSLSRHDHIHHH (SEQ ID NO: 5), and DFTKTSPLGIH (SEQ ID NO: 6).

In certain embodiments, the peptide comprises a member selected from the group consisting of an anti-choline acetyltransferase (anti-ChAT) and anti-calcitonin gene-related peptide.

In certain embodiments, the tissue-binding peptide conjugate comprises a parathyroid-binding conjugate and differentiates parathyroid tissue from other tissue.

In certain embodiments, the peptide comprises a member selected from the group consisting of an anti-parathyroid hormone (PTH) and GATA antibody (e.g., GATA1 antibody, e.g., GATA2 antibody, e.g., GATA3 antibody, e.g., GATA4 antibody, e.g., GATA5 antibody).

In certain embodiments, the anti-PTH targets a PTH protein having a sequence comprising Ser-Val-Ser-Glu-Ile-Gln-Leu-Met-His-Asn-Leu-Gly-Lys-His-Leu-Asn-Ser-Met-Glu-Arg-Val-Glu-Trp-Leu-Arg-Lys-Lys-Leu-Gln-Asp-Val-His-Asn-Phe (SEQ ID NO: 1).

In certain embodiments, the peptide comprises GATA3 antibody.

In certain embodiments, the administering comprises topically administering a solution (e.g., wherein the solution comprises the two or more different probe species) (e.g., wherein the solution comprises the plurality of compositions) (e.g., wherein the solution comprises the formulation).

In certain embodiments, the administering comprises locally depositing the solution to a tissue via a device (e.g., a nano-scaled spray device, e.g., a nebulizer device).

In certain embodiments, the device atomizes the solution of the tissue-binding composition (e.g., as a spray) and dispenses the solution at a low flow rate to the tissue.

In certain embodiments, the low flow rate is in a range from about 1 μL/min to about 100 μL/min (e.g., a range from about 10 μL/min to about 75 L/min, e.g., a range from about 15 μL/min to about 50 μL/min).

In certain embodiments, the method comprises modulating a power supply to modulate a charge of a surface of at least one composition (e.g., nanoparticle surface) in the solution, thereby altering tissue penetration and/or binding properties of the at least one composition.

In another aspect, the invention is directed to a device (e.g., a nano-scaled air-spray, e.g., a nebulizer device) for topical application of the solution, comprising: a capillary tube within a nominally larger tube (e.g., a sprayer); an air or gas pressure source (e.g., wherein the air or gas pressure is controllable); and a pump (e.g., a peristaltic pump, e.g., a syringe pump).

In certain embodiments, the pump is adjustable (e.g., to control a flow rate from about 1 μl/min to about 100 μl/min).

In certain embodiments, the gas pressure source applies a gas pressure in a range from about 1 L/min to about 20 L/min (e.g., from about 1 psi to about 20 psi).

In certain embodiments, the device administers the solution at a temperature (e.g., a controllable temperature) from about 25 degrees C. to about 60 degrees C.

In certain embodiments, an outlet of the larger tube has a diameter within a range from about 80 μm to about 200 μm.

In certain embodiments, a power supply (e.g., wherein the power supply (e.g., low voltage) applies a voltage within a range from about 0 V to about +/−10 V).

Elements of embodiments involving one aspect of the invention (e.g., methods) can be applied in embodiments involving other aspects of the invention, and vice versa.

### Definitions

In order for the present disclosure to be more readily understood, certain terms are first defined below. Additional definitions for the following terms and other terms are set forth throughout the specification.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Administration”: The term “administration” refers to introducing a substance or formulation into a subject. In general, any route of administration may be utilized including, for example, parenteral (e.g., intravenous), oral, topical, subcutaneous, peritoneal, intraarterial, inhalation, vaginal, rectal, nasal, introduction into the cerebrospinal fluid, or instillation into body compartments. In some embodiments, administration is oral. Additionally or alternatively, in some embodiments, administration is parenteral. In some embodiments, administration is intravenous. In certain embodiments, the substance or formulation is administered via local injection vs. IV administration. For example, substances or formulations with peptide-containing compositions (e.g., both particle-containing and non-particle-containing compositions) can be locally injected in a sufficiently high concentration for imaging purposes. In certain embodiments, non-particle peptide-containing compositions are administered via IV.

“Biocompatible”: The term “biocompatible”, as used herein is intended to describe materials that do not elicit a substantial detrimental response in vivo. In certain embodiments, the materials are “biocompatible” if they are not toxic to cells. In certain embodiments, materials are “biocompatible” if their addition to cells in vitro results in less than or equal to 20% cell death, and/or their administration in vivo does not induce inflammation or other such adverse effects. In certain embodiments, materials are biodegradable.

“Biodegradable”: As used herein, “biodegradable” materials are those that, when introduced into cells, are broken down by cellular machinery (e.g., enzymatic degradation) or by hydrolysis into components that cells can either reuse or dispose of without significant toxic effects on the cells. In certain embodiments, components generated by breakdown of a biodegradable material do not induce inflammation and/or other adverse effects in vivo. In some embodiments, biodegradable materials are enzymatically broken down. Alternatively or additionally, in some embodiments, biodegradable materials are broken down by hydrolysis. In some embodiments, biodegradable polymeric materials break down into their component polymers. In some embodiments, breakdown of biodegradable materials (including, for example, biodegradable polymeric materials) includes hydrolysis of ester bonds. In some embodiments, breakdown of materials (including, for example, biodegradable polymeric materials) includes cleavage of urethane linkages.

“Cancer”: As used herein, the term “cancer” refers to a malignant neoplasm or tumor (Stedman's Medical Dictionary, 25th ed.; Hensly ed.; Williams & Wilkins: Philadelphia, 1990). Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B cell ALL, T cell ALL), acute myelocytic leukemia (AML) (e.g., B cell AML, T cell AML), chronic myelocytic leukemia (CML) (e.g., B cell CML, T cell CML), and chronic lymphocytic leukemia (CLL) (e.g., B cell CLL, T cell CLL)); lymphoma such as Hodgkin lymphoma (HL) (e.g., B cell HL, T cell HL) and non Hodgkin lymphoma (NHL) (e.g., B cell NHL such as diffuse large cell lymphoma (DLCL) (e.g., diffuse large B cell lymphoma), follicular lymphoma, chronic lymphocytic leukemia/small lymphocytic lymphoma (CLL/SLL), mantle cell lymphoma (MCL), marginal zone B cell lymphomas (e.g., mucosa associated lymphoid tissue (MALT) lymphomas, nodal marginal zone B cell lymphoma, splenic marginal zone B cell lymphoma), primary mediastinal B cell lymphoma, Burkitt lymphoma, lymphoplasmacytic lymphoma (e.g., Waldenström's macroglobulinemia), hairy cell leukemia (HCL), immunoblastic large cell lymphoma, precursor B lymphoblastic lymphoma and primary central nervous system (CNS) lymphoma; and T cell NHL such as precursor T lymphoblastic lymphoma/leukemia, peripheral T cell lymphoma (PTCL) (e.g., cutaneous T cell lymphoma (CTCL) (e.g., mycosis fungoides, Sezary syndrome), angioimmunoblastic T cell lymphoma, extranodal natural killer T cell lymphoma, enteropathy type T cell lymphoma, subcutaneous panniculitis like T cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES); neuroblastoma; neurofibroma (e.g., neurofibromatosis (NF) type 1 or type 2, schwannomatosis); neuroendocrine cancer (e.g., gastroenteropancreatic neuroendocrine tumor (GEP NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

“Carrier”: As used herein, “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

“Detector”: As used herein, “detector” refers to any detector of electromagnetic radiation including, but not limited to, CCD camera, photomultiplier tubes, photodiodes, and avalanche photodiodes.

“Image”: As used herein, the term “image” is understood to mean a visual display or any data representation that may be interpreted for visual display. For example, a three-dimensional image may include a dataset of values of a given quantity that varies in three spatial dimensions. A three-dimensional image (e.g., a three-dimensional data representation) may be displayed in two-dimensions (e.g., on a two-dimensional screen, or on a two-dimensional printout). The term “image” may refer, for example, to an optical image, an x-ray image, an image generated by: positron emission tomography (PET), magnetic resonance, (MR) single photon emission computed tomography (SPECT), and/or ultrasound, and any combination of these.

“Peptide” or “Polypeptide”: The term “peptide” or “polypeptide” refers to a string of at least two (e.g., at least three) amino acids linked together by peptide bonds. In some embodiments, a polypeptide comprises naturally-occurring amino acids; alternatively or additionally, in some embodiments, a polypeptide comprises one or more non-natural amino acids (i.e., compounds that do not occur in nature but that can be incorporated into a polypeptide chain; see, for example, http://www.cco.caltech.edu/dadgrp/Unnatstruct.gif, which displays structures of non-natural amino acids that have been successfully incorporated into functional ion channels) and/or amino acid analogs as are known in the art may alternatively be employed). In some embodiments, one or more of the amino acids in a protein may be modified, for example, by the addition of a chemical entity such as a carbohydrate group, a phosphate group, a farnesyl group, an isofarnesyl group, a fatty acid group, a linker for conjugation, functionalization, or other modification, etc.

“Radiolabel”: As used herein, “radiolabel” refers to a moiety comprising a radioactive isotope of at least one element. Exemplary suitable radiolabels include but are not limited to those described herein. In some embodiments, a radiolabel is one used in positron emission tomography (PET). In some embodiments, a radiolabel is one used in single-photon emission computed tomography (SPECT). In some embodiments, radioisotopes comprise ^{99m}Tc, ¹¹¹In, ⁶⁴Cu, ⁶⁷Ga, ¹⁸⁶Re, ¹⁸⁸Re, ¹⁵³Sm, ¹⁷⁷Lu, ⁶⁷Cu, ¹²³I, ¹²⁴I, ¹²⁵I, ¹¹C, ¹³N, ¹⁵O, ¹⁸F, ¹⁵³Sm, ¹⁶⁶Ho, ¹⁴⁹Pm, ⁹⁰Y, ²¹³Bi, ¹⁰³Pd, ¹⁰⁹Pd, ¹⁵⁹Gd, ¹⁴⁰La, ¹⁹⁸Au, ¹⁹⁹Au, ¹⁶⁹Yb, ¹⁷⁵Yb, ¹⁶⁵Dy, ¹⁶⁶Dy, ⁶⁷Cu, ¹⁰⁵Rh, ¹¹¹Ag, ⁸⁹Zr, ²²⁵Ac, and ¹⁹²Ir.

“Subject”: As used herein, the term “subject” includes humans and mammals (e.g., mice, rats, pigs, cats, dogs, and horses). In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

“Substantially”: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

“Therapeutic agent”: As used herein, the phrase “therapeutic agent” refers to any agent that has a therapeutic effect and/or elicits a desired biological and/or pharmacological effect, when administered to a subject.

“Treatment”: As used herein, the term “treatment” (also “treat” or “treating”) refers to any administration of a substance that partially or completely alleviates, ameliorates, relives, inhibits, delays onset of, reduces severity of, and/or reduces incidence of one or more symptoms, features, and/or causes of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Drawings are presented herein for illustration purposes, not for limitation.

### BRIEF DESCRIPTION OF DRAWINGS

The foregoing and other objects, aspects, features, and advantages of the present disclosure will become more apparent and better understood by referring to the following description taken in conduction with the accompanying drawings, in which:

FIGS. 1A-1D show topical application of NBP-C′ dots (at 60 μM) to sciatic nerves in mice. Images were acquired with Zeiss Stereo Lumar. V12. Exposure time was 600 ms.

FIGS. 2A-2B show sciatic nerve and muscle fluorescence signal intensity as a function of time (minutes) (FIG. 2A) and sciatic nerve/muscle ratio as a function of time (minutes) (FIG. 2B).

FIGS. 3A-3D show real-time intraoperative nerve mapping in miniswine models using fluorescent C′ dots conjugated with nerve binding peptides.

FIG. 3A shows sciatic nerve exposure for C′ dot applications.

FIG. 3B shows cyclic peptide-bound C′ dots applied to the nerve.

FIG. 3C shows a fluorescent sciatic nerve that is dissected distally.

FIG. 3D shows a sciatic nerve ex vivo with microscopy.

FIGS. 4A-4B shows human facial nerve uptake of cyclic, linear, and

scrambled (control) peptide functionalized C′ dots (15 μM) compared to cyclic peptide-dye conjugates.

FIG. 5A-5B show human ex vivo facial nerve uptake of peptide-Cy5.5 conjugates versus cyclic and scrambled (control) peptide-functionalized-Cy5.5-C′ dots (15 UM).

FIGS. 6A-6C show ex vivo Human Facial Nerve Uptake of NBP-Cy5.5 conjugates versus NBP-C′ dots.

FIGS. 7A-7C show topical application of C′ dot (60 μM) on a mouse facial nerve. Images were acquired with Zeiss Stereo Lum, V12. Exposure time was 600 ms.

FIGS. 8A-8C show images a main trunk and branches of a right facial nerve of a miniswine where 15 μM cyclic NBP-C′ dots were topically applied for 40 minutes.

FIGS. 9A-9B show an excised facial nerve that shows signal extending into the small nerve branches.

FIG. 10A shows an image acquired twenty minutes after administration of ^{99m}Tc-MIBI, before skin incision.

FIG. 10B shows acquisition performed 90 minutes after administration of the radioisotope, after parathyroid excision.

FIG. 10C shows ex vivo imaging of the excised materials.

FIG. 10D shows an image performed after parathyroidectomy and thyroidectomy.

FIG. 11 shows pre-operative PET screening and real-time intraoperative fluorescence-based multiplexed detection of nodal metastases, according to an illustrative embodiment of the invention.

FIG. 12 shows a device comprising a capillary tube within a nominally larger tube (e.g., the sprayer); an air or gas pressure source; a pump; and, as needed, a low voltage-adjustable power supply, according to an illustrative embodiment of the invention. The device can be used to topically apply a solution comprising nanoparticles to a target tissue.

FIG. 13 shows a method for distinguishing lymph nodes and/or lymph node pathways, according to an illustrative embodiment of the invention.

FIG. 14 shows a method for distinguishing one or more nerves, according to an illustrative embodiment of the invention.

FIG. 15 shows a kit comprising containers and at least a first and second probe species and their respective carriers, according to an illustrative embodiment of the invention.

FIG. 16 shows a method for detecting and/or distinguishing light emitted from a first conjugate and a second conjugate, according to an illustrative embodiment of the invention.

### DETAILED DESCRIPTION

Throughout the description, where compositions are described as having, including, or comprising specific components, or where methods are described as having, including, or comprising specific steps, it is contemplated that, additionally, there are compositions of the present invention that consist essentially of, or consist of, the recited components, and that there are methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim.

As described herein, different dyes can be attached to nerve binding peptides and/or incorporated within peptide-functionalized C′ dots to permit fluorescence-based multiplexing for “tagging” various neural structures. The sequence and/or conformation of the cyclic (or linear) peptide used, either in its native form or attached to the particle may be adjusted for different nerve types, to enable visual differentiation of the nerve types during surgery (e.g., the different nerve types have a different color). This is important during various nerve repair surgeries (e.g., surgery for facial droop), where the surgeon tries to find a normal nerve segment (“good side”) to graft to an affected area (“bad side”). Few surgeons can perform these types of surgeries, as it is difficult to differentiate particular types of nerve tissue needed for grafts. The nerve binding peptide (and/or fluorescent particle) compositions would facilitate/simplify such surgeries by allowing visual differentiation of specific nerve tissue types.

Additional applications of the provided nerve-binding peptide conjugates include identification of critical sensory nerves such as the ilioinguinal nerve during inguinal hernia repair. Injury or entrapment of this nerve during surgery can cause disabling chronic pain. Topically apply the described nerve-binding peptide conjugates during this procedure can help provide the surgeon with greater visibility of a nerve that lights up in the operative field which can be avoided.

Furthermore, applications of the provided nerve-binding peptide conjugates extend beyond discriminating between motor and sensory nerves, and also include discriminating between nerves and non-discreet endocrine structures such as parathyroid glands, or other tissue. Parathyroid glands can be difficult to identify and iatrogenic complications related to this surgery would likely be greatly reduced with enhanced visibility provided by the provided nerve-binding peptide conjugates (compared to nerve-binding peptides alone).

In certain embodiments, the conjugated nanoparticles can be applied across the human body (e.g., including spine) in order to provide surgeons with greatly improved visibility of nerves and to discriminate between nerve type and other structures that are difficult to identify. The surgeon is ultimately limited by what he or she can see, and augmenting the surgeon's vision can provide a very significant advance and a new standard of care.

Conjugated nerve binding peptides (NBPs) to C′ dots for targeting/mapping of systemic nerves intraoperatively, while reducing off-target binding to adjacent soft tissue structures, have been described previously by Bradbury et al., International Publication No. WO 2016/100340 published on Jun. 23, 2016. To more selectively discriminate motor and/or sensory nerve branches, new markers, specific for these neural structures, can be conjugated to C′ dots. Thus, for a given nerve, such as the facial nerve, these synthesized particle conjugates can improve the surgeon's ability to distinguish motor from sensory branches.

Furthermore, the provided nerve-binding peptide conjugates can be applied to the operative field, and then irrigated shortly afterward, leaving the conjugated dyes avidly bound to their nerve targets and brightly highlighting sensory and motor nerves in the field. This augmented visibility can greatly increase the safety of parotidectomy.

The following may be utilized for such visual differentiation:
Use of unnatural amino acids such as N-methylated amino acids in the sequence of the peptide (e.g., cyclic or linear);
Use of a peptide (e.g., cyclic or linear) with a secondary structural motif (e.g., alpha-helix structure); and
Use of phage display to increase specificity and differentiate different types of human nerves.

A library of peptide analogues can be developed for particle based detection. Sequence and structural variations can be used to identify optimized nerve binding peptides. Shorter/truncated variants of a parent peptide that exhibit binding properties similar to the full-length 17-residue sequence described in the Appendix can be identified. Linear analogues of NP41 can be synthesized by solid-phase peptide synthesis protocols. Head-to-tail cyclic analogues can be obtained in solution, followed by deprotection and HPLC purification. Different secondary structural motifs (e.g., α-helix), can be assessed using cyclization chemistries.

Phage display approaches can be used for identifying novel human nerve-specific markers. Multiplexing strategy can inform the development of dye-functionalized nerve binding peptide probes, and corresponding particle conjugates, that detect normal nerve tissue markers by chemically adapting (e.g., via cyclization) existing murine nerve binding peptides (NBP) to enhance binding affinity and avidity. Furthermore, phage display can be used to screen for NBP sequences specific to murine nerve tissue, and can be used to identify nerve binding peptide sequences specific to human facial and sciatic nerve tissue specimens, for example.

In addition to harvesting normal nerve segments for treating sites of neural injury (i.e., one side of the face to another), normal nodes can also being harvested from remote sites and transplanted to sites with lymphedema following resection of cancer-bearing nodes. The “lymph node transfer” technique also requires fluorescence-based multiplexing strategies. The following is an example of implementation of this technique for treating lymphedema of the neck following resection of melanoma-bearing nodes. A normal node from the lower abdominal region is preferred. However, nodes in this region may also drain the lower extremity. To avoid taking nodes that drain the lower extremity, two different remote sites in these regions are injected (subcutaneous or subnormal) to distinguish these distributions using the multichannel fluorescent camera system (Artemis Spectrum). One site is injected with cRGDY-PEG-Cy5.5-C′ dots, while the other is injected with cRDGY-PEG-CW800-C′ dots. Nodes seen to drain the lower extremity are not harvested.

Details of various embodiments applicable to the compositions and methods described herein are also provided in, for example, PCT/US14/30401 (WO 2014/145606) by Bradbury et al., PCT/US16/26434 (“Nanoparticle Immunoconjugates”, filed Apr. 7, 2016) by Bradbury et al., PCT/US14/73053 (WO2015/103420) by Bradbury et al., PCT/US15/65816 (WO 2016/100340) by Bradbury et al., PCT/US16/34351 (“Methods and Treatment Using Ultrasmall Nanoparticles to Induce Cell Death of Nutrient-Deprived Cancer Cells via Ferroptosis”, filed May 26, 2016) by Bradbury et al., U.S. 62/330,029 (“Compositions and Methods for Targeted Particle Penetration, Distribution, and Response in Malignant Brain Tumors,” filed Apr. 29, 2016) by Bradbury et al., and U.S. patent application Ser. No. 14/969,877 (“Cyclic Peptides With Enhanced Nerve-Binding Selectivity, Nanoparticles Bound With Said Cyclic Peptides, And Use Of The Same For Real-Time In Vivo Nerve Tissue Imaging, filed Dec. 15, 2015) by Bradbury et al., the contents of which are hereby incorporated by reference in their entireties.

For example, a technique referenced herein as “Reverse Lymphatic Multiplex Mapping (RLMM)” uses ultrasmall nanoparticles (e.g., C dots and/or C′ dots) that fluoresce at two different wavelengths. In certain embodiments, RLMM allows the surgeon to map the lymph nodes which drain the extremities in a manner that visually (e.g., graphically) differentiates them from lymph nodes which drain the tumor site. This enhanced visualization allows the surgeon to avoid damaging critical lymph nodes that may lead to lymphedema. Furthermore, RLMM using these ultrasmall nanoparticles can be used to safely perform vascularized lymph node transplantation in the treatment of lymphedema (e.g., to identify nodes suitable for transplantation). For example, targeted lymph nodes for lymph node harvest draining the trunk can be identified with a nanoparticle using a different colored dye, allowing the surgeon to cherry pick lymph nodes that will not affect drainage of the adjacent limb. This technique allows for the safe harvest of lymph nodes in lymph node transplantation for lymphedema.

As an example, a patient with a particular cancer who needs axillary lymph nodes removed receives a first injection of a first type of C dot that fluoresces at a first spectrally distinct wavelength, where the first injection is injected into or near a tumor site. The patient also receives a second injection of a second type of C dot that fluoresces at a second wavelength spectrally distinct from the first wavelength, where the second injection is injected into an extremity (e.g., an upper or lower extremity near the tumor site) that would be potentially affected by lymphedema if a lymphatic drainage pathway affecting that extremity is disturbed by removal of a lymph node for that pathway. For example, in the case of melanoma, a first injection site can be at the site of melanoma (e.g., on the trunk, abdomen, pelvis) and the second site would be at the potentially affected extremity. For example, in the case of breast cancer, a first injection site can be the thoracic cavity; and in the case of gynecological cancers, a first injection site can be the pelvic area. The second injection would then be in the extremity that would be potentially affected by lymphedema. Being able to differentiate between the first type and second types of C dots reduces risk of lymphedema to the extremity by avoiding removing the drainage lymph node.

For instance, a patient with breast cancer who needs axillary lymph nodes removed has one type of C dot that fluoresces green which is injected into the breast (e.g., wherein the fluorophore is part of the particle itself or is attached to or otherwise associated with the particle). Another C dot that fluoresces blue (or is otherwise visually or spectrally differentiated from the first C dot) is injected into a potentially affected extremity (e.g., the lower or the upper limb), e.g., an extremity near the tumor site. For example, when removing the axillary nodes, the surgeon can specifically remove only green lymph nodes draining the breast and avoid blue lymph nodes draining the upper limb. The imaging technique can be performed as part of a surgical procedure, or it may be performed for pre-surgical imaging. This technique can be performed with any cancer where a node is removed or transplanted.

As another example, RLMM allows the surgeon to reduce the risk in operations involving nerves and consequences of nerve damage, particularly facial nerve damage. For example, a first type of nanoparticle with ligands attached that facilitate (at least temporary) binding of the nanoparticle to motor nerves are administered to a patient, and a second type of nanoparticle with ligands attached that facilitate binding of the nanoparticle to sensory nerves are administered to the patient, wherein the first and second type of nanoparticles are visually (or spectrally) distinguishable from each other. During surgery, motor nerves fluoresce one color (e.g., green) while sensory nerves fluoresce another color (e.g., blue), providing the surgeon with enhanced ability to identify different nerves and/or avoid cutting certain nerves. The technique may be useful in both surgical settings and non-surgical (e.g., pre-surgical imaging) settings.

In certain embodiments, the nanoparticle comprises silica, polymer (e.g., poly(lactic-co-glycolic acid) (PLGA)), biologics (e.g., protein carriers), and/or metal (e.g., gold, iron). In certain embodiments, the nanoparticle is a “C′ dot” or “C′ dot” as described in U.S. Publication No. 2013/0039848 A1 by Bradbury et al., which is hereby incorporated by reference herein in its entirety.

In certain embodiments, the nanoparticle is spherical. In certain embodiments, the nanoparticle is non-spherical. In certain embodiments, the nanoparticle is or comprises a material selected from the group consisting of metal/semi-metal/non-metals, metal/semi-metal/non-metal-oxides, -sulfides, -carbides, -nitrides, liposomes, semiconductors, and/or combinations thereof. In certain embodiments, the metal is selected from the group consisting of gold, silver, copper, and/or combinations thereof.

The nanoparticle may comprise metal/semi-metal/non-metal oxides including silica (SiO₂), titania (TiO₂), alumina (Al₂O₃), zirconia (ZᵣO₂), germania (GeO₂), tantalum pentoxide (Ta₂O₅), NbO₂, etc., and/or non-oxides including metal/semi-metal/non-metal borides, carbides, sulfide and nitrides, such as titanium and its combinations (Ti, TiB₂, TiC, TiN, etc.).

The nanoparticle may comprise one or more polymers, e.g., one or more polymers that have been approved for use in humans by the U.S. Food and Drug Administration (FDA) under 21 C.F.R. § 177.2600, including, but not limited to, polyesters (e.g., polylactic acid, poly(lactic-co-glycolic acid), polycaprolactone, polyvalerolactone, poly(1,3-dioxan-2-one)); polyanhydrides (e.g., poly(sebacic anhydride)); polyethers (e.g., polyethylene glycol); polyurethanes; polymethacrylates; polyacrylates; polycyanoacrylates; copolymers of PEG and poly(ethylene oxide) (PEO).

The nanoparticle may comprise one or more degradable polymers, for example, certain polyesters, polyanhydrides, polyorthoesters, polyphosphazenes, polyphosphoesters, certain polyhydroxyacids, polypropylfumerates, polycaprolactones, polyamides, poly(amino acids), polyacetals, polyethers, biodegradable polycyanoacrylates, biodegradable polyurethanes and polysaccharides. For example, specific biodegradable polymers that may be used include but are not limited to polylysine, poly(lactic acid) (PLA), poly(glycolic acid) (PGA), poly(caprolactone) (PCL), poly(lactide-co-glycolide) (PLG), poly(lactide-co-caprolactone) (PLC), and poly(glycolide-co-caprolactone) (PGC). Another exemplary degradable polymer is poly(beta-amino esters), which may be suitable for use in accordance with the present application.

In certain embodiments, a nanoparticle can have or be modified to have one or more functional groups. Such functional groups (within or on the surface of a nanoparticle) can be used for association with any agents (e.g., detectable entities, targeting entities, therapeutic entities, or PEG). In addition to changing the surface charge by introducing or modifying surface functionality, the introduction of different functional groups allows the conjugation of linkers (e.g., (cleavable or (bio-) degradable) polymers such as, but not limited to, polyethylene glycol, polypropylene glycol, PLGA, etc.), targeting/homing agents, and/or combinations thereof.

The number of ligands attached to the nanoparticle may range from about 1 to about 20, from about 2 to about 15, from about 3 to about 10, from about 1 to about 10, or from about 1 to about 6. The small number of the ligands attached to the nanoparticle helps maintain the hydrodynamic diameter of the present nanoparticle which meet the renal clearance cutoff size range. Hilderbrand et al., Near-infrared fluorescence: application to in vivo molecular imaging, Curr. Opin. Chem. Biol., 14:71-9, 2010.

In certain embodiments, therapeutic agents other than PSMAi may be attached to the nanoparticle. The therapeutic agents include antibiotics, antimicrobials, antiproliferatives, antineoplastics, antioxidants, endothelial cell growth factors, thrombin inhibitors, immunosuppressants, anti-platelet aggregation agents, collagen synthesis inhibitors, therapeutic antibodies, nitric oxide donors, antisense oligonucleotides, wound healing agents, therapeutic gene transfer constructs, extracellular matrix components, vasodialators, thrombolytics, anti-metabolites, growth factor agonists, antimitotics, statin, steroids, steroidal and non-steroidal anti-inflammatory agents, angiotensin converting enzyme (ACE) inhibitors, free radical scavengers, PPAR-gamma agonists, small interfering RNA (siRNA), microRNA, and anti-cancer chemotherapeutic agents. The therapeutic agents encompassed by the present embodiment also include radionuclides, for example, ⁹⁰Y, ¹³¹I and ¹⁷⁷Lu. The therapeutic agent may be radiolabeled, such as labeled by binding to radiofluorine ¹⁸F.

Cancers that may be treated include, for example, any cancer. In certain embodiments, the cancers are melanoma, breast, and gynecologic cancers.

In certain embodiments, a contrast agent may be attached to the present nanoparticle for medical or biological imaging. In certain embodiments may include positron emission tomography (PET), single photon emission computed tomography (SPECT), computerized tomography (CT), magnetic resonance imaging (MRI), optical bioluminescence imaging, optical fluorescence imaging, and combinations thereof. In certain embodiments, the contrast agent can be any molecule, substance or compound known in the art for PET, SPECT, CT, MRI, and optical imaging. The contrast agent may be radionuclides, radiometals, positron emitters, beta emitters, gamma emitters, alpha emitters, paramagnetic metal ions, and supraparamagnetic metal ions. The contrast agents include, but are not limited to, iodine, fluorine, Cu, Zr, Lu, At, Yt, Ga, In, Tc, Gd, Dy, Fe, Mn, Ba and BaSO₄. The radionuclides that may be used as the contrast agent attached to the nanoparticle of the present embodiment include, but are not limited to, ⁸⁹Zr, ⁶⁴Cu, ⁶⁸Ga, ⁸⁶Y, ¹²⁴I, ¹⁷⁷Lu, ²²⁵Ac, ²¹²Pb, and ²¹¹At. Alternatively, a contrast agent may be indirectly conjugated to the nanoparticle, by attaching to linkers or chelators. The chelators may be adapted to bind a radionuclide. The chelators that can be attached to the present nanoparticle may include, but are not limited to, N,N′-Bis(2-hydroxy-5-(carboxyethyl)-benzyl)ethylenediamine-N,N′-diacetic acid (HBED-CC), 1,4,7,10-tetraazacyclododecane-1,4,7,10-tetraacetic acid (DOTA), diethylenetriaminepentaacetic (DTPA), desferrioxamine (DFO) and triethylenetetramine (TETA).

In certain embodiments, a probe species comprises nanoparticles. In certain embodiments, the nanoparticles have a silica architecture and dye-rich core. In certain embodiments, the dye rich core comprises a fluorescent reporter. In certain embodiments, the fluorescent reporter is a near infrared or far red dye. In certain embodiments, the fluorescent reporter is selected from the group consisting of a fluorophore, fluorochrome, dye, pigment, fluorescent transition metal, and fluorescent protein. In certain embodiments, the fluorescent reporter is selected from the group consisting of Cy5, Cy5.5, Cy2, FITC, TRITC, Cy7, FAM, Cy3, Cy3.5, Texas Red, ROX, HEX, JA133, AlexaFluor 488, AlexaFluor 546, AlexaFluor 633, AlexaFluor 555, AlexaFluor 647, DAPI, TMR, R6G, GFP, enhanced GFP, CFP, ECFP, YFP, Citrine, Venus, YPet, CyPet, AMCA, Spectrum Green, Spectrum Orange, Spectrum Aqua, Lissamine and Europium. In certain embodiments, imaging is performed in normal lighting settings. In certain embodiments, imaging is performed with some to zero levels of ambient lighting settings.

The imaging methods herein can be used with a number of different fluorescent probe species (or, as in embodiments using a tandem bioluminescent reporter/fluorescent probe, the fluorescent species thereof), for example, (1) probes that become activated after target contact (e.g., binding or interaction) (Weissleder et al., Nature Biotech., 17:375-378, 1999; Bremer et al., Nature Med., 7:743-748, 2001; Campo et al., Photochem. Photobiol. 83:958-965, 2007); (2) wavelength shifting beacons (Tyagi et al., Nat. Biotechnol., 18:1191-1196, 2000); (3) multicolor (e.g., fluorescent) probes (Tyagi et al., Nat. Biotechnol., 16:49-53, 1998); (4) probes that have high binding affinity to targets, e.g., that remain within a target region while non-specific probes are cleared from the body (Achilefu et al., Invest. Radiol., 35:479-485, 2000; Becker et al., Nature Biotech. 19:327-331, 2001; Bujai et al., J. Biomed. Opt. 6:122-133, 2001; Ballou et al. Biotechnol. Prog. 13:649-658, 1997; and Neri et al., Nature Biotech 15:1271-1275, 1997); (5) quantum dot or nanoparticle-based imaging probes, including multivalent imaging probes, and fluorescent quantum dots such as amine T2 MP EviTags® (Evident Technologies) or Qdot® Nanocrystals (Invitrogen™); (6) non-specific imaging probes e.g., indocyanine green, AngioSense® (VisEn Medical); (7) labeled cells (e.g., such as cells labeled using exogenous fluorophores such as VivoTag™ 680, nanoparticles, or quantum dots, or by genetically manipulating cells to express fluorescent or luminescent proteins such as green or red fluorescent protein; and/or (8) X-ray, MR, ultrasound, PET or SPECT contrast agents such as gadolinium, metal oxide nanoparticles, X-ray contrast agents including iodine based imaging agents, or radioisotopic form of metals such as copper, gallium, indium, technetium, yttrium, and lutetium including, without limitation, 99m-Tc, 111-In, 64-Cu, 67-Ga, 186-Re, 188-Re, 153-Sm, 177-Lu, and 67-Cu. The relevant text of the above-referenced documents are incorporated by reference herein. Another group of suitable imaging probes are lanthanide metal-ligand probes. Fluorescent lanthanide metals include europium and terbium. Fluorescence properties of lanthanides are described in Lackowicz, 1999, Principles of Fluorescence Spectroscopy, 2^{nd} Ed., Kluwar Academic, New York, the relevant text incorporated by reference herein. In the methods of this embodiment, the imaging probes can be administered systemically or locally by injecting an imaging probe or by topical or other local administration routes, such as “spraying”. Furthermore, imaging probes used in the embodiment of this invention can be conjugated to molecules capable of eliciting photodynamic therapy. These include, but are not limited to, Photofrin, Lutrin, Antrin, aminolevulinic acid, hypericin, benzoporphyrin derivative, and select porphyrins. In particular, fluorescent probe species are a preferred type of imaging probe. A fluorescent probe species is a fluorescent probe that is targeted to a biomarker, molecular structure or biomolecule, such as a cell-surface receptor or antigen, an enzyme within a cell, or a specific nucleic acid, e.g., DNA, to which the probe hybridizes. Biomolecules that can be targeted by fluorescent imaging probes include, for example, antibodies, proteins, glycoproteins, cell receptors, neurotransmitters, integrins, growth factors, cytokines, lymphokines, lectins, selectins, toxins, carbohydrates, internalizing receptors, enzyme, proteases, viruses, microorganisms, and bacteria.

In certain embodiments, probe species have excitation and emission wavelengths in the red and near infrared spectrum, e.g., in the range 550-1300 or 400-1300 nm or from about 440 to about 1100 nm, from about 550 to about 800 nm, or from about 600 to about 900 nm. Use of this portion of the electromagnetic spectrum maximizes tissue penetration and minimizes absorption by physiologically abundant absorbers such as hemoglobin (<650 nm) and water (>1200 nm). Probe species with excitation and emission wavelengths in other spectrums, such as the visible and ultraviolet light spectrum, can also be employed in the methods of the embodiments of the present invention. In particular, fluorophores such as certain carbocyanine or polymethine fluorescent fluorochromes or dyes can be used to construct optical imaging agents, e.g. U.S. Pat. No. 6,747,159 to Caputo et al. (2004); U.S. Pat. No. 6,448,008 to Caputo et al. (2002); U.S. Pat. No. 6,136,612 to Della Ciana et al. (2000); U.S. Pat. No. 4,981,977 to Southwick, et al. (1991); U.S. Pat. No. 5,268,486 to Waggoner et al. (1993); U.S. Pat. No. 5,569,587 to Waggoner (1996); U.S. Pat. No. 5,569,766 to Waggoner et al. (1996); U.S. Pat. No. 5,486,616 to Waggoner et al. (1996); U.S. Pat. No. 5,627,027 to Waggoner (1997); U.S. Pat. No. 5,808,044 to Brush, et al. (1998); U.S. Pat. No. 5,877,310 to Reddington, et al. (1999); U.S. Pat. No. 6,002,003 to Shen, et al. (1999); U.S. Pat. No. 6,004,536 to Leung et al. (1999); U.S. Pat. No. 6,008,373 to Waggoner, et al. (1999); U.S. Pat. No. 6,043,025 to Minden, et al. (2000); U.S. Pat. No. 6,127,134 to Minden, et al. (2000); U.S. Pat. No. 6,130,094 to Waggoner, et al. (2000); U.S. Pat. No. 6,133,445 to Waggoner, et al. (2000); U.S. Pat. No. 7,445,767 to Licha, et al. (2008); U.S. Pat. No. 6,534,041 to Licha et al. (2003); U.S. Pat. No. 7,547,721 to Miwa et al. (2009); U.S. Pat. No. 7,488,468 to Miwa et al. (2009); U.S. Pat. No. 7,473,415 to Kawakami et al. (2003); also WO 96/17628, EP 0 796 111 B1, EP 1 181 940 B1, EP 0 988 060 B1, WO 98/47538, WO 00/16810, EP 1 113 822 B1, WO 01/43781, EP 1 237 583 A1, WO 03/074091, EP 1 480 683 B1, WO 06/072580, EP 1 833 513 A1, EP 1 679 082 A1, WO 97/40104, WO 99/51702, WO 01/21624, and EP 1 065 250 A1; and Tetrahedron Letters 41, 9185-88 (2000).

Exemplary fluorochromes for probe species include, for example, the following: Cy5.5, Cy5, Cy7.5 and Cy7 (GER Healthcare); AlexaFluor660, AlexaFluor680, AlexaFluor790, and AlexaFluor750 (Invitrogen); VivoTag™680, VivoTag™-S680, VivoTag™-S750 (VISEN Medical); Dy677, Dy682, Dy752 and Dy780 (Dyomics®); DyLight® 547, and/or DyLight® 647 (Pierce); HiLyte Fluor™ 647, HiLyte Fluor™ 680, and HiLyte Fluor™ 750 (AnaSpec®); IRDye® 800CW, IRDye® 800RS, and IRDye® 700DX (Li-Cor®); ADS780WS, ADS830WS, and ADS832WS (American Dye Source); XenoLight CF™ 680, XenoLight CF™ 750, XenoLight CF™ 770, and XenoLight DiR (Caliper® Life Sciences); and Kodak® X-SIGHT® 650, Kodak® X-SIGHT 691, Kodak® X-SIGHT 751 (Carestream® Health).

Suitable means for imaging, detecting, recording or measuring the present nanoparticles may also include, for example, a flow cytometer, a laser scanning cytometer, a fluorescence micro-plate reader, a fluorescence microscope, a confocal microscope, a bright-field microscope, a high content scanning system, and like devices. More than one imaging techniques may be used at the same time or consecutively to detect the present nanoparticles. In one embodiment, optical imaging is used as a sensitive, high-throughput screening tool to acquire multiple time points in the same subject, permitting semi-quantitative evaluations of tumor marker levels. This offsets the relatively decreased temporal resolution obtained with PET, although PET is needed to achieve adequate depth penetration for acquiring volumetric data, and to detect, quantitate, and monitor changes in receptor and/or other cellular marker levels as a means of assessing disease progression or improvement, as well as stratifying patients to suitable treatment protocols.

The compositions and methods described herein can be used with other imaging approaches such as the use of devices including but not limited to various scopes (microscopes, endoscopes), catheters and optical imaging equipment, for example computer based hardware for tomographic presentations.

In certain embodiments, the methods can be used in the detection, characterization and/or determination of the localization of a disease, especially early disease, the severity of a disease or a disease-associated condition, the staging of a disease, and monitoring and guiding various therapeutic interventions, such as surgical procedures, and monitoring and/or development of drug therapy and delivery, including cell based therapies. In certain embodiments, the methods can also be used in prognosis of a disease or disease condition. With respect to each of the foregoing, examples of such disease or disease conditions that can be detected or monitored (before, during or after therapy) include inflammation (for example, inflammation caused by arthritis, for example, rheumatoid arthritis), cancer (for example, any cancer, e.g., melanoma, breast, and gynecologic cancers, including metastases), central nervous system disease (for example, a neurodegenerative disease, such as Parkinson's disease or Alzheimer's disease, Huntington's Disease, amyotrophic lateral sclerosis, prion disease), inherited diseases, metabolic diseases, environmental diseases (for example, lead, mercury and radioactive poisoning, skin cancer), neurodegenerative disease, and surgery-related complications (such as graft rejection, organ rejection, alterations in wound healing, fibrosis or other complications related to surgical implants). In certain embodiments, the methods can therefore be used, for example, to determine the presence of tumor cells and localization and metastases of tumor cells, the presence and localization of inflammation, including the presence of activated macrophages, for instance in atherosclerosis or arthritis, the presence and localization of vascular disease including areas at risk for acute occlusion (e.g., vulnerable plaques) in coronary and peripheral arteries, regions of expanding aneurysms, unstable plaque in carotid arteries, and ischemic areas, and stent thrombosis.

Embodiments presented herein include, for example, use of an in vivo imaging system to evaluate cancer (e.g., breast cancer, metastatic melanoma) by visualizing different tumor lymphatic drainage pathways and nodal distributions following local injection of probe species. Real-time and simultaneous intraoperative visualization of peripheral nerves and nodal disease in prostate cancer, and other cancers, can be performed using targeted dual-modality probe species. The targeted dual-modality probe species localizes to the nodes. The wavelength of emitted light from each probe species discriminates between the nodes that are to be removed or the nodes that are not to be removed. For example, the first probe species may have an emission wavelength of about 700 nm while the second probe species has an emission wavelength of about 800 nm. The real-time and simultaneous visualization for intraoperative visualization of nerves can also be conducted for parotid tumors, and for tumors of the larynx for mapping laryngeal nerves.

In certain embodiments, the methods and systems are used to evaluate nodal metastases by visualizing different tumor lymphatic drainage pathways and nodal distributions following local injection. Simultaneous multicolor platforms can be visualized in real-time using the handheld Artemis fluorescence camera system. For example, real-time optical imaging using the Artemis™ handheld fluorescent camera system can be used, along with different NIR dye-containing silica nanoparticles, to simultaneously map different nodal distributions.

In certain embodiments, the methods and systems are performed/used to visualize intraoperatively in real-time nerves and nodal for nerve transplants using targeted dual-modality silica nanoparticles. Intraoperative visualization and detection tools will improve post-surgical outcomes in patients, enabling complete resection without functional damage to adjacent neuromuscular structures (i.e., nerves). To achieve this end, translatable, dual-modality silica nanoparticles (NPs) can improve targeted disease localization pre-operatively, as well as enhance real-time visualization of prostatic nerves, nodal disease, and residual prostatic tumor foci or surgical margins using a handheld NIR fluorescence camera system. Further information can be found in U.S. Publication No. US 2015/0182118 A1 (Appendix C), whose contents of which are hereby incorporated by reference in its entirety.

The methods differ from previous methods in their ability to carry out simultaneous detection of light signals at different wavelengths in real-time for treatment of lymphedema and nerve (e.g., motor vs. sensory) transplantation. In certain embodiments, the method comprises a multichannel fluorescence camera system that simultaneously detects multiple wavelengths from multiple dyes in real-time. In certain embodiments, the imaging apparatus comprises a hand-held fluorescent imaging system that uses multiple detectors and associated circuitry that can collect distinguishable signals from the multiple types of probe species with higher signal-to-noise ratio. In certain embodiments, the system does not distinguish multiple signal types received at a single detector with optical time division multiplexing, as do other previous imaging systems.

### EXAMPLES

### Conjugation of Peptides to C′ Dots for Visual Differentiation of Nerve Tissue During Surgical Procedures

The peptide used in the present Examples is 17 AA NP41, which includes the core sequence NTQTLAKAPEHT (SEQ ID NO: 3). However, the present Examples are not limited to the provided 17 AA nerve binding peptide. For example, other peptides (e.g., an anti-parathyroid hormone (PTH) and GATA antibody (e.g., GATA1 antibody, e.g., GATA2 antibody, e.g., GATA3 antibody, e.g., GATA4 antibody, e.g., GATA5 antibody), e.g., anti-ChAT, e.g., anti-CGRP) can be used in various embodiments, as described herein.

Choline acetyltransferase (ChAT), the enzyme catalyzing the formation of acetylcholine, is overexpressed in motor nerves, such as the facial nerve. Choline acetyltransferase therefore serves as an attractive target for motor neurons.

### Choline Acetyltransferase

Commercially available anti-ChAT antibody fragments (e.g., scFv or Fab) were used as ligands for creating C′ dot immunoconjugates for motor nerve mapping. Antibody fragments were reacted with N-Acetyl-L-cysteine NHS ester (1:10 molar ratio) in PBS buffer overnight (pH=7.5), and subsequently purified by a bio-gel column. The resulting purified antibody fragment, which bears a cysteine residue, was then added to MAL-PEG-C′ dots; the latter particle conjugate incorporating a maleimide functional group on its surface. Conjugation reactions were performed in PBS buffer (pH=7.5) with 1:5 molar ratio of particle to antibody fragment. The product was purified using gel permeation chromatography and a Sephadex column. C′ dots were synthesized to encapsulate several near-infrared dyes (e.g., Cy5.5) for intraoperative visualization.

### Calcitonin Gene-Related Peptide

Calcitonin gene-related peptide (CGRP), a 37-amino acid neuropeptide, is abundant in sensory neurons, and therefore serves as an attractive target for identifying this nerve type.

Commercially available anti-CGRP antibody scFv fragment was utilized for conjugation to C′ dots. The anti-CGRP antibody scFv fragment was reacted with N-Acetyl-L-cysteine NHS ester (1:10 molar ratio) in PBS buffer overnight (pH=7.5), followed by purification with a bio-gel column. The purified CGRP antibody fragment was then conjugated overnight to MAL-PEG-C′ dots in PBS buffer (pH=7.5) with 1:5 molar ratio (particle:fragment). Additional purification was performed with GPC and Sephadex column. C′ dots were synthesized to encapsulate a different near-infrared dye (i.e., cw800) from that used for motor nerves to enhance neural discrimination.

### Protocols for Applying C′ Dot Conjugates to Nerves

Ex vivo experiments were performed using human nerve tissue samples. The tissue samples used were cadaveric facial nerve and facial sural nerve freshly excised and obtained by the National Disease Research Interchange (NDRI). Tissue was prepared on 24-well plates, washed with PBS, and then incubated with 15 μM C′ dot conjugates, along with controls, at room temperature for 30 minutes. C′ dot conjugate concentrations were determined using a fluorescence plate reader. After incubation with particle conjugates for about 20-30 minutes, tissue samples were subjected to several rounds of washing with PBS. The plates were imaged using an IVIS Spectrum imaging system. Region of interest (ROI) analyses of fluorescence signal are performed for both nerve and muscle specimens using PerkinElmer software.

Miniswine surgical studies were performed to evaluate C′ dot conjugate binding to facial and sciatic nerves. Facial nerves were exposed intraoperatively and particle conjugates were topically applied at concentrations ranging from 15 M-60 μM. After incubating nerve specimens for about 30 minutes, phosphate buffered saline was used to wash the exposed site. Images and video were acquired by a hand-held camera system to read out fluorescence intensity and track particle diffusion along the nerve segment. To validate C′ dot distribution and localization in nerve tissues, nerve specimens were harvested from mini-swine, flash-frozen in OCT, cut in cross section (10 μm thickness) and prepared on slides for microscopy.

### Sciatic Nerve: In Vivo Topical Administration (Murine and Miniswine Studies)

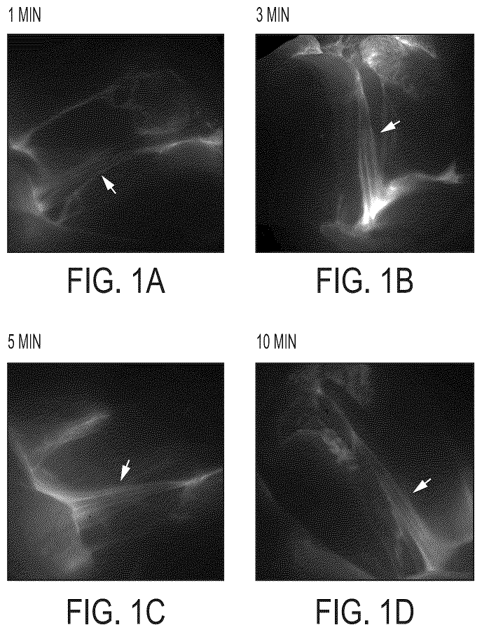

FIGS. 1A-1D show topical application of nerve binding peptide (NBP)-C′ dots (at 60 μM) to sciatic nerves in mice. Images were acquired with Zeiss Stereo Lumar. V12. Exposure time was 600 ms. 60 μM of 17 amino acid (AA) cyclic-peptide conjugated C′ dots was applied on sciatic nerve of nude mice. C′ dots were incubated for 1, 3, 5, and 10 minutes, followed by three times of PBS buffer washing. Zeiss stereo lumar scope was used to observe the strength and distribution of fluorescence signal. Mice were kept under isoflurane anesthesia during surgery.

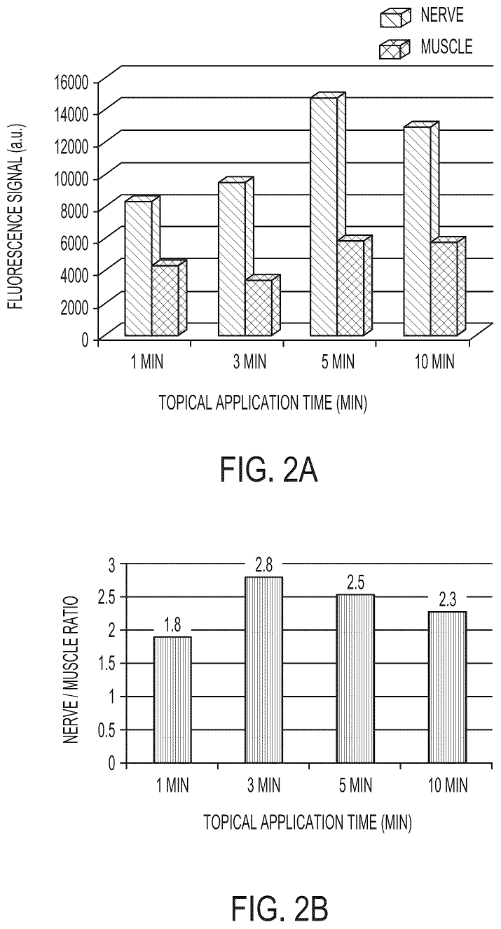

FIGS. 2A-2B show sciatic nerve and muscle fluorescence signal intensity as a function of time (minutes) (FIG. 2A) and sciatic nerve/muscle ratio as a function of time (minutes) (FIG. 2B). 60 μM of 17AA-cyclic-peptide conjugated C′ dots was topically applied to the proximal portions of sciatic nerves in normal nude mice over a 10 min time interval (e.g., 1, 3, 5, 10 min), followed by three PBS washes. A Zeiss stereo lumar scope was used to observe the intensity and distribution of fluorescence signal along the nerves. Mice were maintained under isoflurane anesthesia during surgery. Region-of-interest analyses were placed over areas of high fluorescence signal on the nerve, as well as in the surrounding tissue (e.g., muscle) to generate nerve-to-background or nerve-to-muscle ratios over time. The highest nerve/muscle ratio was found to be ˜3 at around 3 minutes post-incubation.

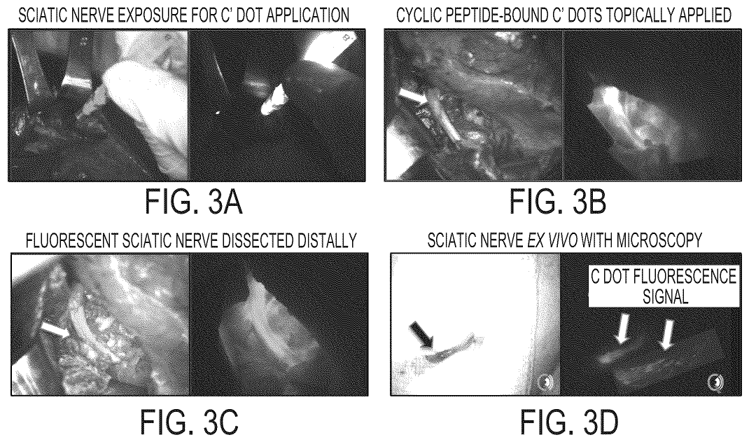

FIGS. 3A-3D show real-time intraoperative nerve mapping in miniswine models using fluorescent C′ dots adapted with nerve binding peptides. FIG. 3A shows sciatic nerve exposure for C′ dot applications. FIG. 3B shows cyclic peptide-bound C′ dots applied to the nerve. FIG. 3C shows a fluorescent sciatic nerve that is dissected distally. FIG. 3D shows a sciatic nerve ex vivo with microscopy.

### Facial Nerve: Three Ex Vivo Topical Experiments

As described herein, ratios (e.g., range of values) are provided: cyclic peptide-C′ dots to cyclic peptide alone: from about 2 to about 6; and cyclic peptide-C′ dots to scrambled peptide-C′ dot: from about 3 to about 6.

### Experiment #1

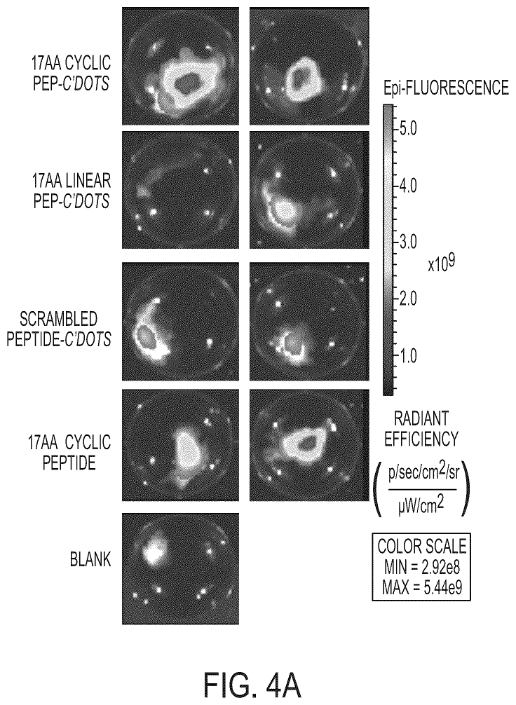

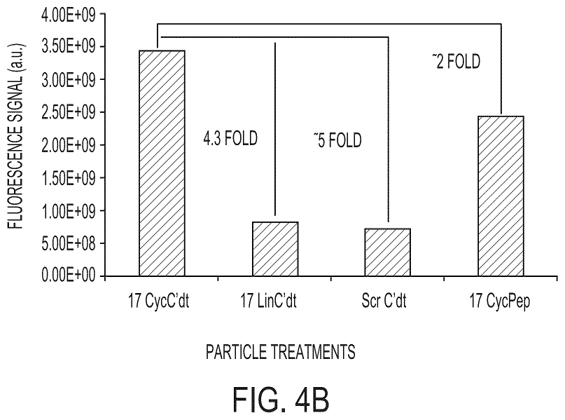

FIGS. 4A-4B shows human facial nerve uptake of cyclic, linear, and scrambled (control) peptide functionalized C′ dots (15 μM) compared to cyclic peptide-dye conjugates. Ex vivo binding/uptake studies comparing peptide-dye (Cy5.5) conjugates to peptide-functionalized deep red/NIR dye-containing (Cy5.5) C′ dots for human nerve specimens were performed. Human facial nerve was sectioned into 0.5 cm length fragments and incubated in 15 μM solutions of peptides or peptide-bound C′ dots for 40 minutes at room temperature followed by multiple phosphate buffered saline washings. Non-invasive region of interest (ROI) analyses obtained 40-min post-incubation by IVIS Spectrum imaging and demonstrated the highest-to-lowest optical signal in nerve tissue exposed to peptide-bound C′ dots as follows: the signal detected using 17AA-cyclic peptide-bound C′ dots was greater than the signal detected using 17-AA cyclic peptide which was greater than the signal using 17-AA linear peptide-bound C′ dots which was greater than the signal using scrambled cyclic peptide-bound C′ dots.

### Experiment #2

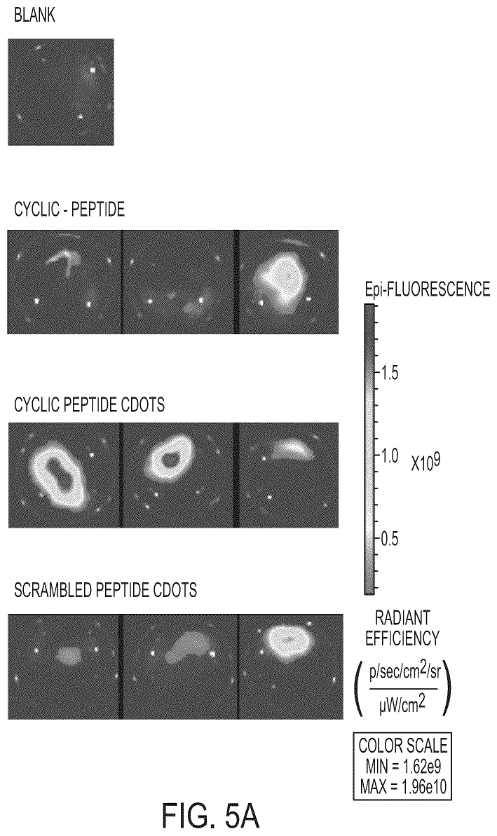

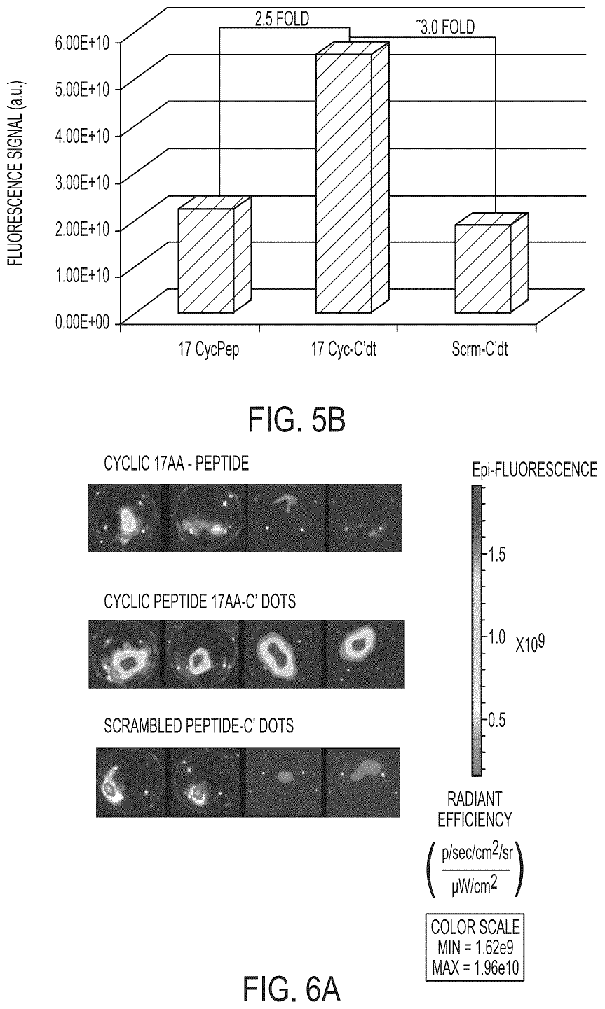

FIG. 5A-5B show human ex vivo facial nerve uptake of peptide-Cy5.5 conjugates versus cyclic and scrambled (control) peptide-functionalized-Cy5.5-C′ dots (15 μM). Ex vivo binding/uptake studies comparing peptide-dye conjugates to peptide-functionalized deep red/NIR dye-containing (Cy5.5) C′ dots for human nerve specimens. Human facial nerve was sectioned into 0.5 cm length fragments and incubated in 15 μM solutions of peptides or peptide-bound C′ dots for 40 minutes with slightly shaking at room temperature, followed by three phosphate buffer saline washes. Region of interest analyses were obtained 40 minutes post-incubation by IVIS Spectrum imaging; highest-to-lowest optical signal was found as follows: the signal detected from cyclic peptide-bound C′ dots was greater than the signal detected from cyclic peptide was greater than the signal detected from scrambled peptide-bound C′ dots.

### Experiment #3

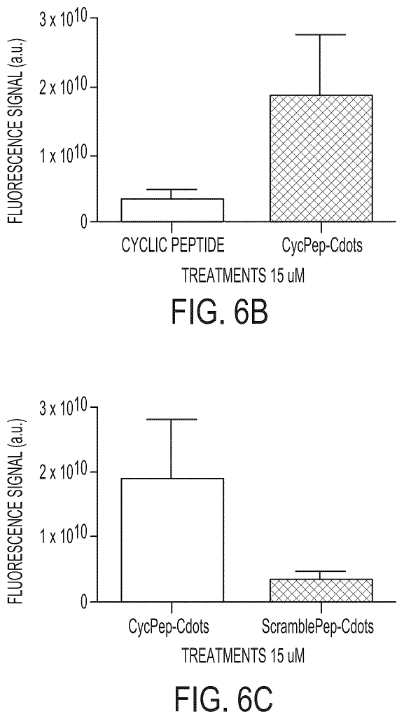

FIGS. 6A-6C show ex vivo human facial nervu Uptake of NBP-Cy5.5 conjugates versus NBP-C′ dots. The Cyclic Peptide-bound C′ dots to Cyclic Peptide ratio was about 6, and the Cyclic Peptide-bound C′ dots to Scrambled peptide-bound C′ dots ratio was also about 6.

### Facial Nerve: In Vivo Topical (Murine Studies)

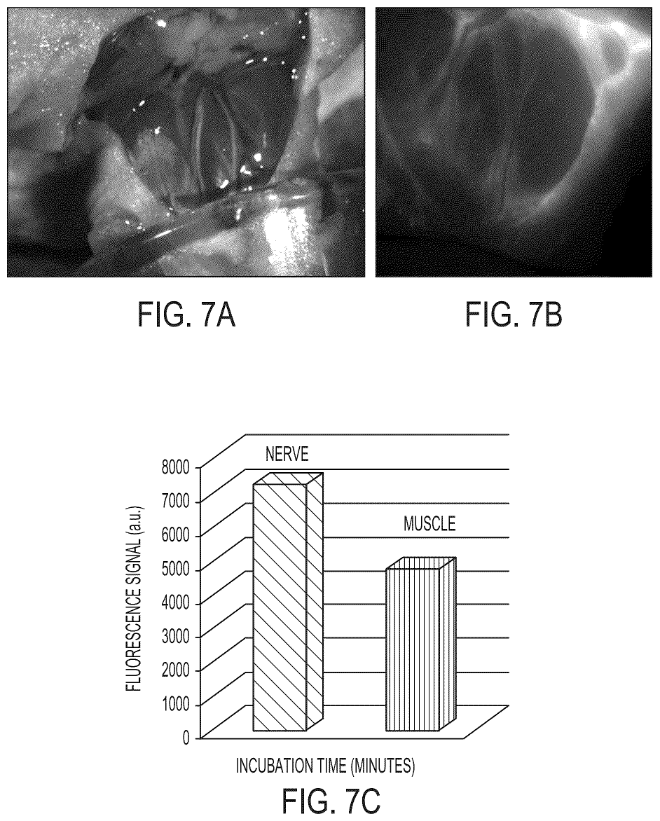

FIGS. 7A-7C show topical application of C′ dot (60 μM) on a mouse facial nerve. Images were acquired with Zeiss Stereo Lum, V12. Exposure time was 600 ms. 60 μM of 17 cyclic-peptide conjugated C′ dots was applied on facial nerve of nude mice. C′ dots were incubated for 3 minutes, followed by three times of PBS buffer washing. Zeiss stereo lumar scope was used to observe the strength and distribution of fluorescence signal. Mice were kept under isoflurane anesthesia during surgery. ROIs on nerve or surrounding muscle were obtained and compared its fluorescence intensity via fluorescence results images taken from Zeiss stereo lumar scope. Facial nerve to muscle ratio was about 1.5.

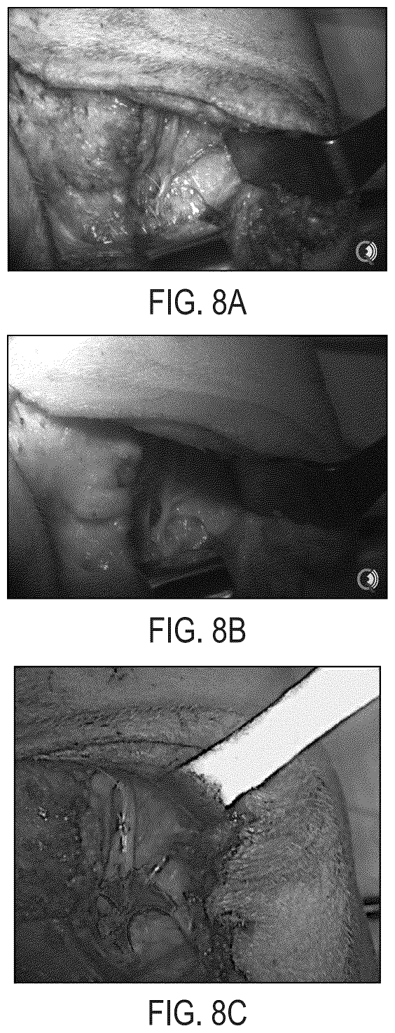

FIGS. 8A-8C show images a main trunk and branches of a right facial nerve of a miniswine where 15 μM cyclic NBP-C′ dots were topically applied for 40 minutes. The main trunk and branches of the right facial nerve were dissected and exposed (arrows) Topical application of 15 μM cyclic NBP-C′ dots on the trunk and branches nerve were applied for 40 min followed by multiple washes with PBS. Detection of optical signal involving the nerve and its branches was performed.

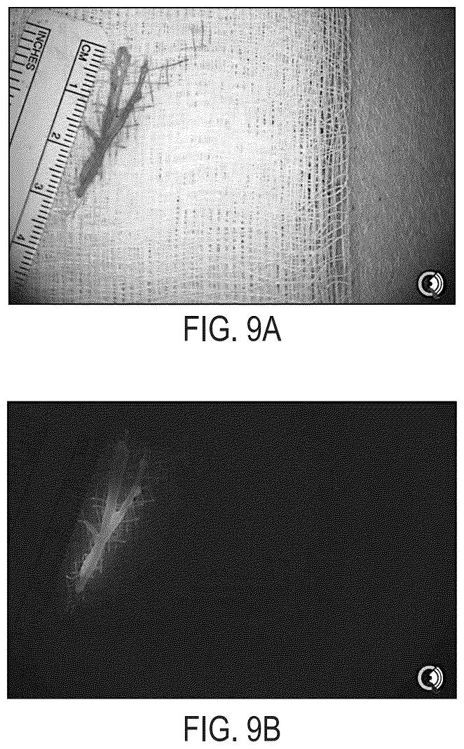

FIGS. 9A-9B show an excised facial nerve that shows signal extending into the small nerve branches.

### Fluorescent Nanoparticles for Parathyroid Optical Identification

Thyroidectomies are very frequent procedures (about 15/week at MSKCC). Incidences of papillary thyroid carcinoma overdiagnosises have increased over the past decade. For example, the most feared complications are recurrent laryngeal nerve lesion and hypoparathyroidism.

Normal parathyroids are very small (from about 5 to about 6 mm in their largest dimension and weigh about 50 mg). Normal parathyroids can be hard to differ from fat or lymph nodes.

Dual-phase scintigraphy with ^{99m}Tc methoxy isobutyl isonitrile (MIBI) is the most commonly used method to identify parathyroid adenomas (success rate 68-86%). MIBI is a lipophilic compound that can be radiolabeled with ^{99m}Tc. After IV administration, the radiopharmaceutical is rapidly and passively accumulated within mitochondria of metabolically active cells. After the injection of ^{99m}Tc-MIBI, its retention is prolonged in parathyroid hyperfunctioning lesions, whereas MIBI is washed out more rapidly from normal thyroid tissue. Retention is related to oxyphilic cells (rich in mitochondria).

Dual-Phase Protocol acquires planar images 15 min and 1-3 hours after the injection. Tracer retention is dependent on several factors such as mitochondria content, cell cycle, and expression of P-glycoprotein efflux protein. SPECT are performed from 10 to 60 min after injection of ^{99m}Tc-MIBI. The use of SPECT/CT fusion images improves the sensitivity of parathyroid imaging in comparison to planar scintigraphy.

### Intraoperative Localization Using a Portable Gamma Probe has Become More Widespread in Minimally Invasive Parathyroid Surgery

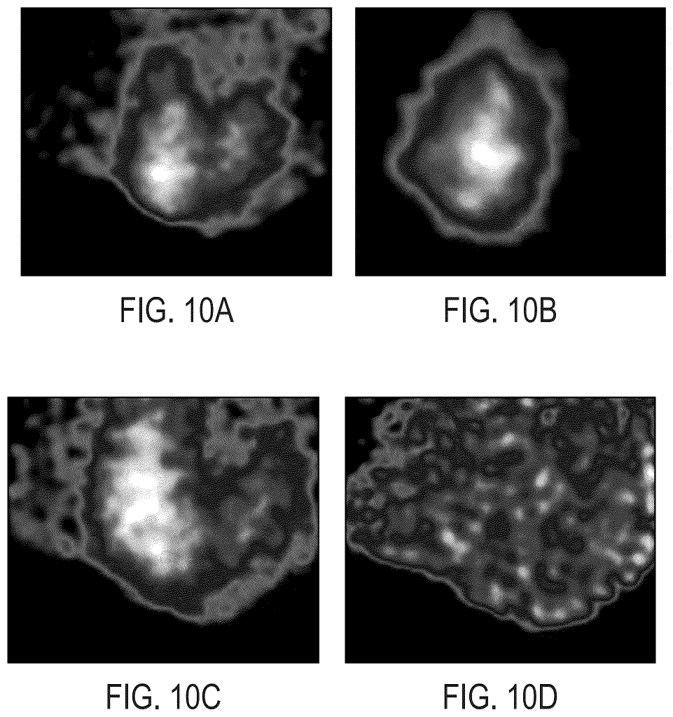

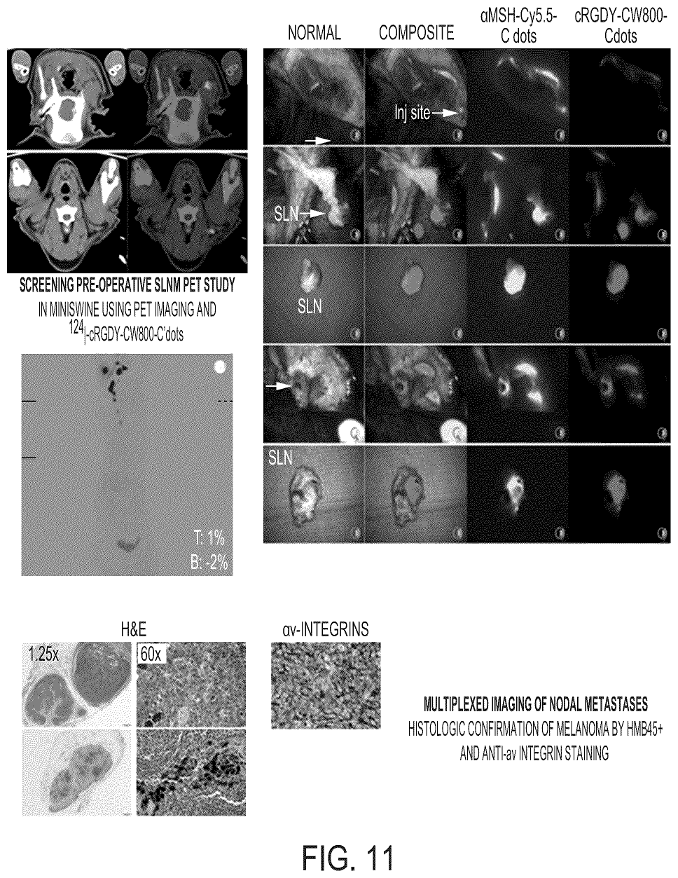

In the operation room, after anesthesia, the nuclear medicine physician administered an intravenous dose of 185 MBq (5mCi) of ^{99m}Tc-MIBI. Four scintigraphic images (FIGS. 10A-10D) of the neck were acquired by placing the collimator at a distance of 15 cm (giving a 20×20 cm field of view): Before skin incision; 15-20 min after the injection; After pathologic parathyroid location; After gland excision; and Ex vivo.

MIBI provides some advantages, including MIBI is already used in vivo and is a small compound. However, MIBI does have limitations, including: specificity (thyroid nodules can also be hot), MIBI is more useful for adenomas (where there are more oxyphilic cells), and even 90 minutes after resection, the thyroid maintains its brightness (FIG. 10B).

As described herein, Anti-Pth can be used to target parathyroids. PTH is synthesized as a precursor protein (presequence of 25 amino acids and prosequence of 6 amino acids). The mature form of PTH comprises 84 amino acids. PTH is almost exclusively produced by parathyroid glands. Regulated by extracellular concentration of calcium-calcium-sensing receptor of the parathyroid glands.

In certain embodiments, the PTH (1-34) Sequence (human) is: Ser-Val-Ser-Glu-Ile-Gln-Leu-Met-His-Asn-Leu-Gly-Lys-His-Leu-Asn-Ser-Met-Glu-Arg-Val-Glu-Trp-Leu-Arg-Lys-Lys-Leu-Gln-Asp-Val-His-Asn-Phe (SEQ ID NO: 1) (www.phoenixpeptide.com).

In certain embodiments, the PTH (1-34) Sequence (rat) is: Ala-Val-Ser-Glu-Ile-Gln-Leu-Met-His-Asn-Leu-Gly-Lys-His-Leu-Ala-Ser-Val-Glu-Arg-Met-Gln-Trp-Leu-Arg-Lys-Lys-Leu-Gln-Asp-Val-His-Asn-Phe (SEQ ID NO: 2) (www.phoenixpeptide.com).

As described herein, a GATA antibody (e.g., GATA1 antibody, e.g., GATA2 antibody, e.g., GATA3 antibody, e.g., GATA4 antibody, e.g., GATA5 antibody) can be used to target parathyroids. GATA proteins have two zinc finger DNA binding domains, Cys-X2-C-X17-Cys-X2-Cys (ZNI and ZNII) that recognize the sequences (A/T) GATA (A/G).

In certain embodiments, GATA3 antibody (www.scbt.com; http://biocare.net (GATA-3 [L50-823]) is used to target parathyroids. GATA3 antibody targets GAT3. Also known as GATA-binding protein 3 and trans-acting T-cell specific factor, GATA3 is a member of the transcription factors that binds the DNA sequence (A/T) GATA (A/G). GATA3 plays an important role in vertebrate embryogenesis. GATA3 is required in promoting and directing cell proliferation, development, and differentiation in many cell types. GATA3 is also involved in the embryonic development of the parathyroid glands and in adult parathyroid cell proliferation. GATA3 protein comprises 443 amino acids.

In the study (Value of GATA3 Immunostaining in the Diagnosis of Parathyroid Tumors), HG3-31 anti-GATA3 mouse monoclonal antibody was used. All 5 normal parathyroid glands, 10 parathyroid hyperplastic glands, 22 parathyroid adenomas, and 6 parathyroid carcinomas were GATA3 positive. All 38 thyroid tumors, 32 renal cell carcinomas, 14 thymic epithelial tumors, and 11 lung carcinoid tumors were GATA3 negative.

GATA3 can be expressed in breast carcinomas (47-100%), urothelial carcinomas (67-93%), and paragangliomas (78%). Rarely expressed in SCC (16-33%) and endometrial adenocarcinomas (˜2%).

In certain embodiments, parathyroid gland markers can be multiplexed in order to distinguish between multiple structures, including node nerves and normal tissue structure.

### Pre-Operative PET Screening and Real-Time Intraoperative Fluorescence-Based Multiplexed Detection of Nodal Metastases

FIG. 1 shows pre-operative PET screening and real-time intraoperative fluorescence-based multiplexed detection of nodal metastases. FIG. 1 shows dual-modality pre-operative and intraoperative imaging of nodal metastases in a spontaneous melanoma miniswine model peritumorally injected with 124I-cRGDY-CW800-C′ dots. High resolution PET scanning demonstrates PET-avid nodes that were subsequently marked for resection intraoperatively. Using a handheld multichannel fluorescence camera system and spectrally-distinct particle probes targeting different receptors (integrin: red; MC1R: green), tumor lymphatic drainage to metastatic nodes was observed in real time with histologic correlation. Simultaneous differential uptake by nodes (yellow color) was found, suggesting sensitivity to detecting various degrees of tumor burden in each of the nodes.

### Device for Topically Applying Tissue-Binding Peptide Conjugate Solution to a Tissue

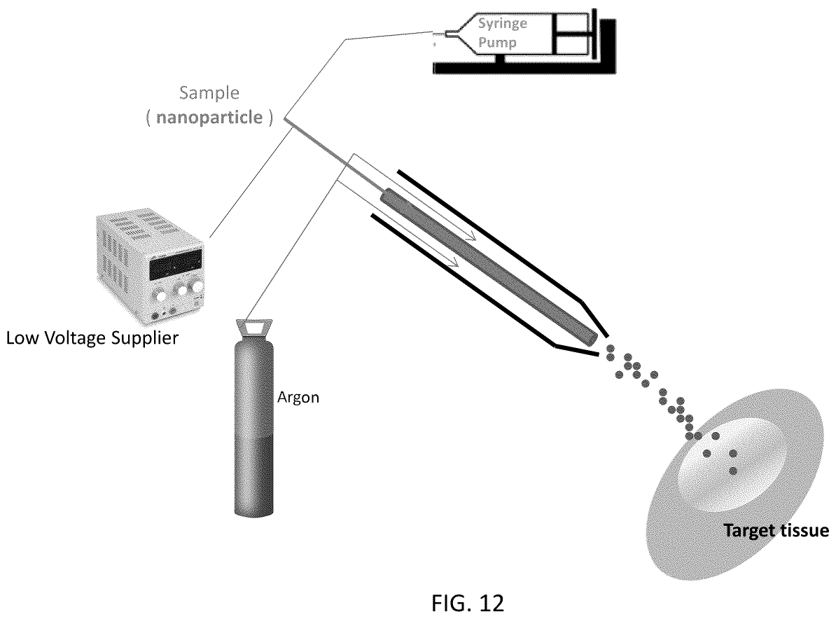

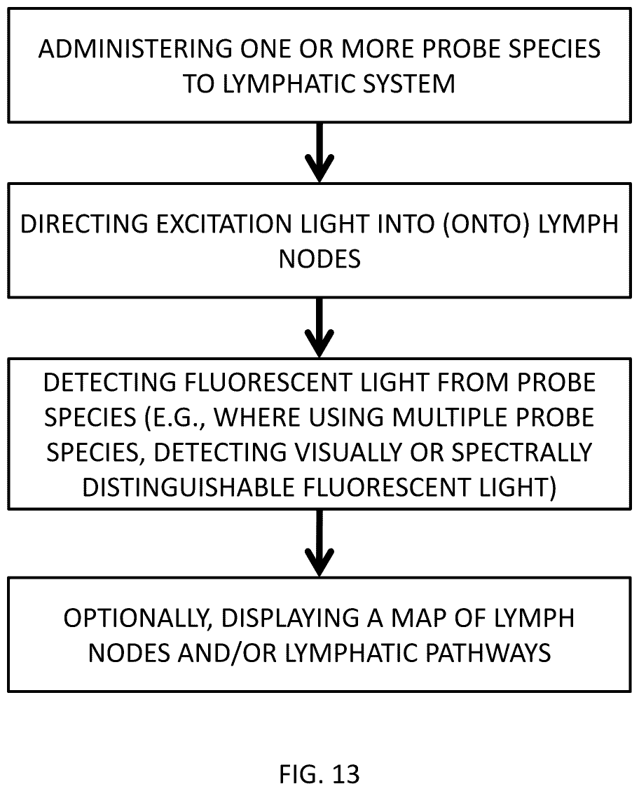

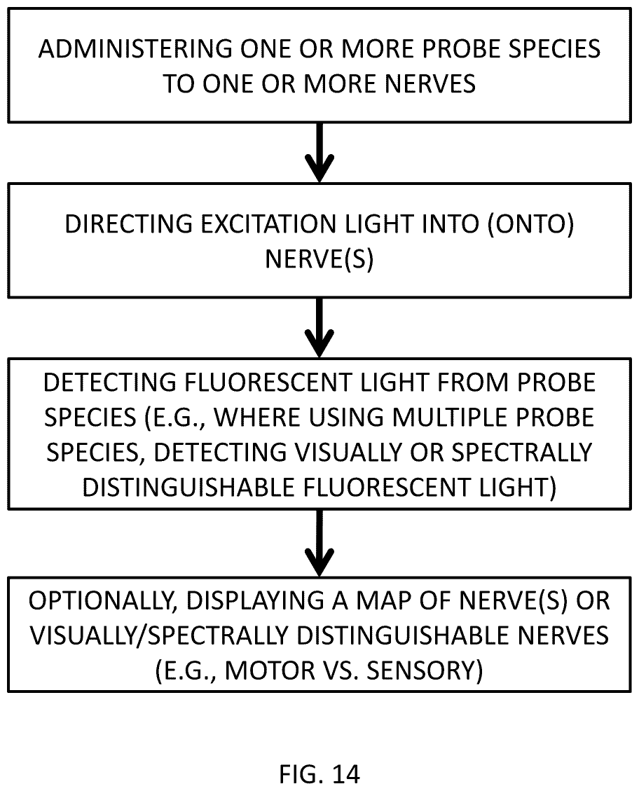

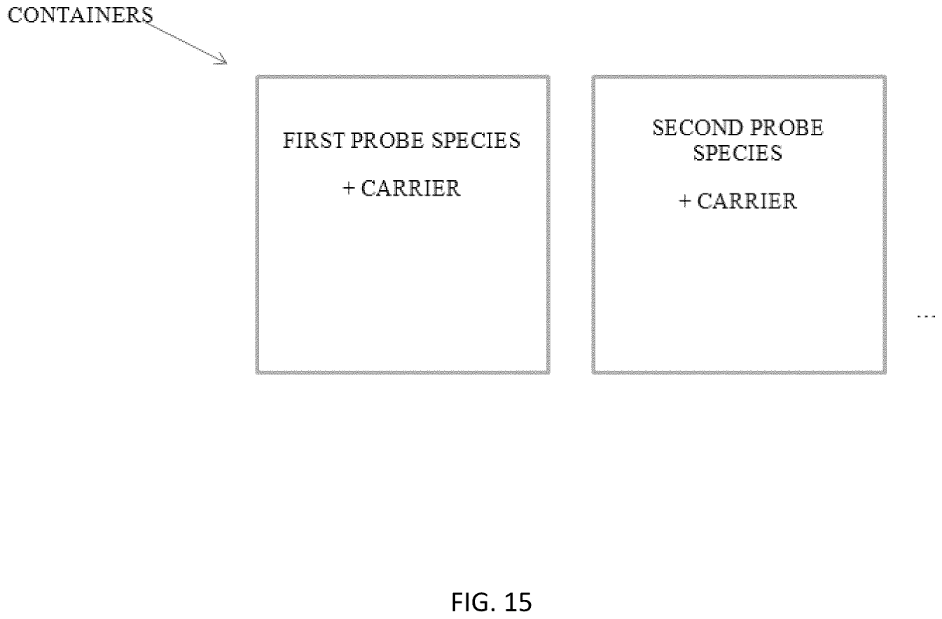

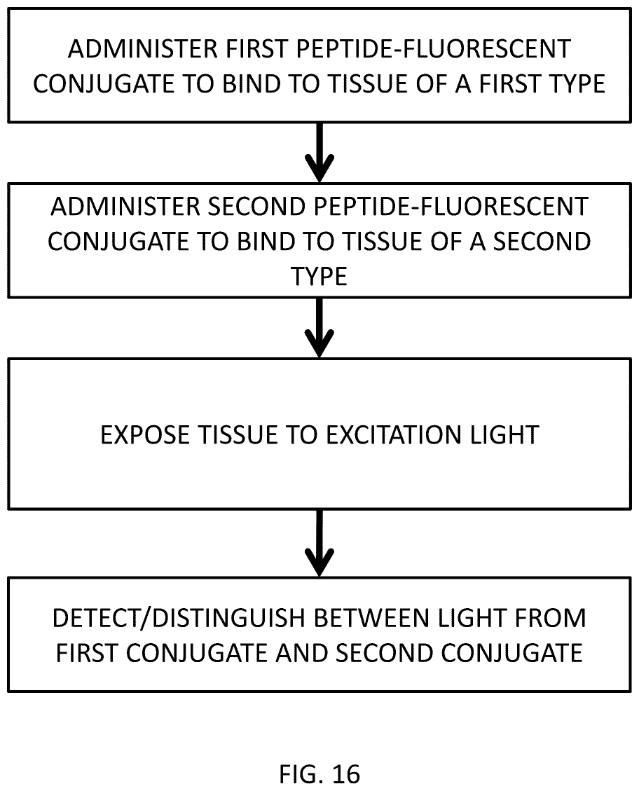

Precise and controlled topical application of the provided nanoparticles to a tissue (e.g., nerve, e.g., lymph node, e.g., parathyroid) of interest in the surgical bed can be achieved through the use of a special co-axial air-spray or nebulizer device (FIG. 12). In certain embodiments, the device comprises: a capillary tube within a nominally larger tube (e.g., the sprayer); an air or gas pressure source; a pump; and, as needed, a low voltage-adjustable power supply. The nanoparticle solution is pumped through the capillary tube, while Argon gas is pumped into the outer sleeve. The flow rate of the nanoparticle solution and the gas pressure can each be regulated. Additionally, the temperature of the solution, gas, or sprayer can be adjusted as needed; the voltage of the sprayer can also be adjusted. These features result in a fine and highly controlled spray, thereby allowing precise topical application of the nanoparticle to the surgical area. In certain embodiments, the device is similar to nebulizers used in electrospray ionization mass spectrometry instruments.

In certain embodiments, surface charge of the nanoparticle compositions can be modulated, thereby affecting surface properties of the nanoparticle compositions. Improved properties of the nanoparticle compositions include increased binding to and penetration of a nerve.

In certain embodiments, the peristaltic or syringe pump controls flow rates have a range from about 1 μl/min to about 100 μl/min. In certain embodiments, gas pressures are in a range from about 1 L/min to about 20 L/min (e.g., from about 1 psi to about 20 psi). In certain embodiments, the temperature is from about 25 degrees C. to about 60 degrees C. In certain embodiments, the spray outlet has a diameter within a range from about 80 μm to about 200 μm. In certain embodiments, the power supply (e.g., low voltage) applies a voltage that has a range from about 0 V to about +/−10 V. In certain embodiments, charge can be added to the nanoparticle compositions to alter penetration and tissue (e.g., nerve, e.g., parathyroid, e.g., lymph node) binding properties.

1.-89. (canceled)

90. A method comprising:
administering two or more different probe species, each comprising a spectrally differentiable fluorescent reporter, to a lymphatic system of a human subject with a tumor, wherein a first probe species is administered to a tumor site of the subject, and a second probe species is administered to an extremity of the subject;
directing excitation light into lymph nodes of the subject, thereby exciting the fluorescent reporters having spectrally distinguishable emission wavelengths;
identifying a lymph node that drains the tumor site, and identifying another lymph node that drains the extremity, using a multichannel fluorescence camera system that simultaneously detects the spectrally distinguishable emission wavelengths in real-time; and
displaying in real-time the fluorescent reporters having spectrally distinguishable emission wavelengths to graphically differentiate between the lymph node that drains the extremity and the lymph node that drains the tumor site,
wherein the two or more different probe species each comprise a silica nanoparticle, and
wherein the silica nanoparticle has a diameter less than 10 nm.

91. The method of claim 90, wherein the tumor site comprises a member selected from the group consisting of a breast, a trunk, an abdomen, a pelvis, and a thoracic cavity.

92. The method of claim 90, wherein the extremity comprises a member selected from the group consisting of an upper limb and a lower limb.

93. The method of claim 90, wherein the identifying comprises simultaneously detecting fluorescent light of the spectrally different emission wavelengths, the detected fluorescent light having been emitted by the fluorescent reporters of the respective probe species in the lymph nodes and/or drainage pathways as a result of illumination by the excitation light so as to discriminate between signals received from each probe species.

94. The method of claim 90, wherein the fluorescent reporter of the first probe species having received the excitation light fluoresces at a spectrally distinguishable wavelength compared to the fluorescent reporter of the second probe species having received the excitation light.

95. The method of claim 93, further comprising identifying a lymph node for excision.

96. The method of claim 90, further comprising displaying a map of lymph nodes and/or lymphatic pathways of the lymphatic system, wherein the map graphically differentiates between specific lymph nodes and/or between specific lymph node types.

97. The method of claim 96, wherein the tumor site comprises a member selected from the group consisting of a breast, a trunk, an abdomen, a pelvis, and a thoracic cavity.

98. The method of claim 90, wherein the multichannel fluorescence camera system is a handheld multichannel fluorescence camera system.

99. A method comprising:
administering two or more different probe species, each comprising a spectrally differentiable fluorescent reporter, to a lymphatic system of a human subject, wherein a first probe species is administered to a trunk of the subject, and a second probe species is administered to an extremity of the subject;
directing excitation light into lymph nodes of the subject, thereby exciting the fluorescent reporters having spectrally distinguishable emission wavelengths;
identifying a lymph node in the trunk for harvesting in transplantation for lymphedema, and identifying another lymph node that drains the extremity, using a multichannel fluorescence camera system that simultaneously detects the spectrally distinguishable emission wavelengths in real-time; and
displaying in real-time the fluorescent reporters having spectrally distinguishable emission wavelengths to graphically differentiate between the lymph node that drains the extremity and the lymph node for harvesting,
wherein the two or more different probe species each comprise a silica nanoparticle, and
wherein the silica nanoparticle has a diameter less than 10 nm.

100. The method of claim 99, wherein the extremity comprises a member selected from the group consisting of an upper limb and a lower limb.

101. The method of claim 99, wherein the identifying comprises simultaneously detecting fluorescent light of the spectrally different emission wavelengths, the detected fluorescent light having been emitted by the fluorescent reporters of the respective probe species in the lymph nodes and/or drainage pathways as a result of illumination by the excitation light so as to discriminate between signals received from each probe species.

102. The method of claim 99, wherein the fluorescent reporter of the first probe species having received the excitation light fluoresces at a spectrally distinguishable wavelength compared to the fluorescent reporter of the second probe species having received the excitation light.

103. The method of claim 99, further comprising displaying a map of lymph nodes and/or lymphatic pathways of the lymphatic system, wherein the map graphically differentiates between specific lymph nodes and/or between specific lymph node types.
